(19) United States
(12) Patent Application Publication
(10) Pub. No.: US 2024/0407734 A1
(43) Pub. Date: Dec. 12, 2024

(54) METHODS AND SYSTEMS FOR DISEASE PREDICTION

(71) Applicant: Dexcom, Inc., San Diego, CA (US)

(72) Inventors: Jee Hye PARK, San Diego, CA (US); Spencer Troy FRANK, San Diego, CA (US); David A. PRICE, San Diego, CA (US); Charles R. STROYECK, San Diego, CA (US); Arunachalam PANCH SANTHANAM, San Diego, CA (US); Joseph J. BAKER, San Diego, CA (US); Peter C. SIMPSON, San Diego, CA (US); Kazanna C. HAMES, San Diego, CA (US); Qi AN, San Diego, CA (US); Abdulrahman JBAILY, San Diego, CA (US); Justin Yi-Kai LEE, San Diego, CA (US); Stephanie Grace MOORE, San Diego, CA (US)

(21) Appl. No.: 18/737,588

(22) Filed: Jun. 7, 2024

Related U.S. Application Data
(60) Provisional application No. 63/616,365, filed on Dec. 29, 2023.
(60) Provisional application No. 63/506,791, filed on Jun. 7, 2023.

(51) Int. Cl. A61B 5/00; A61B 5/145; G16H 50/20

(52) U.S. Cl. CPC A61B 5/7275; A61B 5/0022; A61B 5/14532; A61B 5/7246; A61B 5/7267; G16H 50/20

(57) ABSTRACT

A method for predicting disease is provided. The method includes generating biased analyte data by adding analyte sensor bias to historical analyte data, associating the biased analyte data with clinical disease diagnoses associated with the historical analyte data, and extracting features from the biased analyte data. The method further includes, for each model of a number of models, generating disease predictions based on different combinations of the features extracted from the biased analyte data, and evaluating the disease predictions based on the clinical disease diagnoses associated with the biased analyte data. The method further includes selecting a model and a combination of features based on a performance metric and a robustness metric.

### CROSS-REFERENCE TO RELATED APPLICATIONS

This application claims the benefit of U.S. Provisional Application Ser. Nos. 63/506,791 (filed on Jun. 7, 2023) and 63/616,365 (filed on Dec. 29, 2023), the contents of which are incorporated by reference herein in their entirety.

### BACKGROUND

Certain medical conditions, such as diabetes, liver disease, kidney disease, heart disease, etc., may produce or alter a concentration of one or more analytes in the blood and, in many cases, the interstitial fluid under the surface (epidermis) of the skin. An analyte is simply a chemical substance, compound, molecule, element, etc., that may be identified or measured, such as glucose (with the chemical formula C₆H₁₂O₆), lactate (the conjugate base of lactic acid with the chemical formula C₃H₅O₃⁻), potassium (the chemical element K), troponin (a complex of three regulatory proteins, i.e., troponin C, I and T), etc. The body's inability to regulate and manage these analytes is because of, in many cases, the presence of a variety of diseases, including diabetes, liver disease, kidney disease, heart disease, etc.

Diabetes is a metabolic disease that occurs, inter alia, when the pancreas does not produce and release enough insulin into the bloodstream to maintain normal blood glucose levels. Insulin reduces blood glucose levels by allowing cells in the muscles, liver and adipose tissue to absorb glucose and use it (or store it) as a source of energy. When observed continuously and over time, a patient's glucose levels may provide an indication of diabetes, such as prediabetes, Type 1 or Type 2 diabetes, gestational diabetes mellitus (GDM), etc.

Liver disease causes progressive deterioration, damage and loss of function of the liver, and includes chronic liver disease (CLD) as well as genetic disorders. When observed continuously and over time, a patient's lactate levels may provide an indication of chronic liver disease, such as nonalcoholic fatty liver disease (NAFLD), nonalcoholic steatohepatitus (NASH), etc.

Kidney disease causes progressive deterioration, damage and loss of function of the kidneys. Kidney disease, such as acute kidney disease, chronic kidney disease (CKD), etc., may eventually progress to end-stage renal disease (ESRD) at which time the kidneys no longer function on their own. When observed continuously and over time, a patient's potassium levels may provide indications of chronic kidney disease.

Heart disease, which is one type of cardiovascular disease (CVD), causes progressive deterioration, damage and loss of function of the heart. When observed continuously and over time, a patient's levels of troponin, creatinine phosphokinase (CPK), or other cardiac enzymes, may provide an indication of heart disease, such as coronary heart disease, acute coronary syndrome (ACS), myocardial ischemia, etc.

Other medical conditions may also alter healthy concentrations of one or more analytes in the blood and interstitial fluid of the body, such as sleep apnea affecting the oxygen concentration in the blood, etc.

### BRIEF DESCRIPTION OF THE DRAWINGS

FIG. 1 illustrates aspects of an example health management system, in accordance with embodiments of the present disclosure.

FIGS. 2A, 2B, and 2C illustrate aspects of an example continuous analyte monitoring (CAM) system, in accordance with embodiments of the present disclosure.

FIG. 3 illustrates example input data and metric data for use by the health management system of FIG. 1, in accordance with embodiments of the present disclosure.

FIG. 4 depicts a block diagram of an example computer device, in accordance with embodiments of the present disclosure.

FIG. 5A depicts an example process flow diagram for evaluating, selecting and training a model and a combination of analyte features for predicting disease, in accordance with embodiments of the present disclosure.

FIG. 5B depicts another example process flow diagram for evaluating and selecting a model and a combination of analyte features for predicting disease, in accordance with embodiments of the present disclosure.

FIGS. 6A and 6B present measured glucose data for a person clinically diagnosed with diabetes and a person not clinically diagnosed with diabetes, respectively, in accordance with embodiments of the present disclosure.

FIG. 6C presents an autocorrelation mean feature for a person clinically diagnosed with diabetes and a person not clinically diagnosed with diabetes, in accordance with embodiments of the present disclosure.

FIG. 6D presents a set point frequency feature for a person clinically diagnosed with diabetes and a person not clinically diagnosed with diabetes, in accordance with embodiments of the present disclosure.

FIG. 7A depicts an example artificial neural network (ANN), in accordance with embodiments of the present disclosure.

FIG. 7B depicts an example logistic regression (LR) model, in accordance with embodiments of the present disclosure.

FIG. 8 depicts an example process flow diagram representing operations for predicting disease, in accordance with embodiments of the present disclosure.

FIGS. 9A and 9B depict an example graphical user interface (GUI) for displaying measured analyte data and a disease prediction on a display device, in accordance with embodiments of the present disclosure.

FIG. 10A depicts an example process flow diagram representing operations for evaluating and selecting a model and a combination of analyte features for predicting disease, in accordance with embodiments of the present disclosure.

FIG. 10B depicts an example process flow diagram representing operations for training a model based on a combination of analyte features to predict disease, in accordance with embodiments of the present disclosure.

FIG. 11 depicts an example process flow diagram representing operations for predicting disease, in accordance with embodiments of the present disclosure.

### DETAILED DESCRIPTION

Generally, conventional tests administered to screen for, and diagnose, many medical conditions have a variety of weaknesses that often lead to improper diagnosis. Many conventional tests are often inaccurate because a given test administered to a person on different days may result in inconsistent diagnoses due to various external factors causing analyte levels to fluctuate, such as sickness, stress, increased exercise, pregnancy, etc. For example, even though the HbA1c test measures an average glucose level over the previous two to three months, the HbA1c test results are greatly impacted by the person's blood glucose levels in the weeks leading up the test. As such, HbA1c test results can be greatly affected by changes in blood properties during the three-month time period, such as due to illness or pregnancy.

Many conventional tests often have poor concordance. In other words, different conventional tests do not necessarily detect the same medical condition in the same person. This lack of consistency between different conventional tests may lead to an inaccurate diagnosis or a failure to determine a proper treatment plan. For example, a person may have a high fasting glucose level but an HbA1c score within the normal range. In these situations, different doctors may reach different conclusions regarding whether the person has diabetes as well as the treatment recommendation for the person. Another issue with conventional tests is reproducibility—repeating the same conventional test may produce different results for the same person, such as an Oral Glucose Tolerance Test (OGTT).

Additionally, administering conventional tests to a broad spectrum of people often presents a variety of drawbacks, such as the requirement to visit a doctor's office or a lab in order to give a blood sample, etc. Physical or psychological barriers may also arise that prevent people from undergoing conventional testing, which reduces or eliminates the benefits associated with early disease detection. For example, many of the conventional tests for diabetes require the person to be in a fasted state, which can be difficult, or even dangerous, for some users, including pregnant women.

Embodiments of the present disclosure advantageously predict a disease for a user based on continuous analyte concentration level measurements.

In certain embodiments, analyte concentration levels are measured by a continuous analyte monitoring (CAM) system worn by the user, a combination of features is determined from the measured analyte data, and a disease prediction is generated based on the combination of features. The disease prediction may be generated by a model that has been selected and trained for classification, categorization and/or prediction tasks, such as rule-based models, generalized linear models, artificial neural networks (ANNs), logistic regression (LR) models, polynomial regression (PR) models, etc. The model development process generally includes evaluating disease predictions based on different combinations of features from historical analyte data that have been adjusted to simulate sensor variability (or bias), and selecting a model and a combination of features for training based on a performance metric and a robustness metric.

Accurately predicting the disease and notifying the user, doctor, health care provider, telemedicine service, etc., may prevent serious damage to the user's heart, blood vessels, eyes, kidneys, nerves, etc., as well as death due to the disease.

The CAM system includes an analyte sensor and a sensor electronics module (SEM). In certain embodiments, the analyte sensor is configured to be inserted through the user's skin to measure one or more interstitial analyte concentration levels relevant to predicting one or more diseases over an observation period. In certain embodiments, the CAM system may also include a temperature sensor, and the SEM may correct the measured analyte concentration levels based on the temperature measured by the temperature sensor. The SEM is configured to generate sensor data packages that include measured analyte data or temperature-corrected measured analyte data, and transmit the sensor data packages to a computing device, such as a mobile computing device (display device), etc. Unlike conventional tests, which are point-in-time tests and traditionally administered in a lab or doctor's office, the CAM system measures analyte concentration levels continuously, and in an ambulatory manner, over the course of the observation period while the user is at home, at work, etc. It is, therefore, the continuous nature of the CAM system that allows for enough analyte data to be gathered about a user, which in turn enables the disease prediction and diagnosis techniques described herein. In other words, it would be challenging, if not impossible, to implement the technical improvements described herein using point-in-time analyte measurements.

As described above, a variety of models may be used for diagnosis prediction, including rule-based models and machine learning (ML) models. A description of example implementations of such models is provided below. Once trained, the models are configured to receive measured analyte data and provide disease predictions.

Generally, a model may be trained using historical analyte data and historical outcome data of a user population to predict a particular disease for an individual user. The historical outcome data may include clinical disease diagnoses that indicate whether each user of the population has been clinically diagnosed with the particular disease based on one or more independent sources, such as sources that did not consider the historical analyte data. For example, the historical analyte data may be generated by CAM systems worn by each user, and the historical outcome data for each user is then associated with the historical analyte data for each user.

In many cases, the historical analyte data may include systemic inaccuracies due to analyte sensor manufacturing variabilities which cause sensor-to-sensor, or lot-to-lot, differences in their respective measured analyte concentration levels. More specifically, manufacturing variabilities for analyte sensors, such as sensor bias, etc., typically exist between sensor lots, sensor models, sensor manufacturers, etc., thereby leading to sensor-to-sensor differences in measurement accuracy. In other words, the same analyte concentration level in the interstitial fluid may generate different measured analyte concentration levels in different analyte sensors. As a result, if historical analyte data are exclusively relied upon during the development of a model, the model may incorrectly predict the disease for some users due to these systemic inaccuracies.

Accordingly, to solve the technical problem described above, certain embodiments described herein provide a technical solution involving adding analyte sensor bias to the historical analyte data to generate biased analyte data, and the biased analyte data may be associated with the same historical outcome data as the historical analyte data for model development purposes. More particularly, a number of models may be evaluated using the biased analyte data to determine the relative performance of each model with respect to different amounts of sensor manufacturing variabilities that are introduced into the historical analyte data, as discussed below. Additionally, instead of using the biased analyte data exclusively, features representing trends, patterns, relationships, etc., may be extracted from the biased analyte data for both model development and disease prediction purposes. Generally, certain features may be advantageously insensitive to variations in the concentration levels of the biased analyte data, and may be identified during model development, as discussed below.

Examples of features include trend-related features (such as autocorrelation and autocorrelation mean features, etc.), time-related and day-related features (such as day features, time of day features, etc.), variability and stability features (such as rate of change features, etc.), frequency-related features (such as set point frequency and baseline features, etc.), analyte concentration level (measured analyte data value) features (such as peaks features, standard analyte features, etc.

As discussed, a variety of models may be used for disease prediction. For example, in certain embodiments, rule-based models may be used to predict disease. Generally, a rule-based model uses a set of rules to analyze data. These rules are sometimes referred to as conditional statements or “If-Then” statements as they tend to follow the line of “If X Then Y.” More particularly, a set of rules (such as a set of conditional or If-Then statements) may be used to predict the disease.

In certain embodiments, the rule set may be created based on empirical research or analysis of historical patient records, such as records stored in a historical records database. In some cases, the reference library may become very granular. For example, other factors in the reference library may be used to create the rule set, such as gender, age, diet, disease history, family disease history, body mass index (BMI), etc. Increased granularity may provide more accurate disease predictions using rule-based models. Generally, the rule set may be stored in a reference library.

In certain embodiments, ML models may be used to predict disease, such as linear regression (LR) models, etc. Generally, the selection of the model and the combination of features involves performing multiple variability simulations over a number of simulation rounds, in which the historical analyte data are adjusted to simulate sensor variability (i.e., the biased analyte data).

More particularly, in certain embodiments, one or more models may be evaluated using different combinations of features (feature combinations) extracted from the biased analyte data, such as two features, three features, four features, etc. For each model, disease predictions are generated based on different combinations of features extracted from the biased analyte data, and the disease predictions are evaluated based on the historical outcome data associated with the biased analyte data. The model and combination of features that produces the best prediction performance with respect to the biased analyte data are selected based on a performance metric and a robustness metric.

The performance metric indicates the prediction accuracy of each feature, and the robustness metric indicates the insensitivity of each feature to the simulated sensor variabilities. Advantageously, the combination of features may be selected to effectively balance the performance metric and the robustness metric, such as selecting one or more features with high performance metric and one or more features with a robustness metric that meets the required robustness criteria.

The selected combination of features are then extracted from the historical analyte data, and the selected model is trained based on the selected combination of features and the clinical disease diagnoses associated with the historical analyte data. In other words, the model is trained based on the historical analyte data without the addition of analyte sensor bias.

During an observation or diagnostic period, the CAM system measures analyte concentration levels of a user and generates measured analyte data, the selected combination of features is determined from the measured analyte data, and a disease prediction for the user is generated based on the selected combination of features. The disease prediction may be displayed to the user on a display device. In certain embodiments, the disease prediction may be stored in a user database for access by a doctor, health care provider, telemedicine service, etc., using a mobile or network computing device. Other information may also be presented, such as visualizations of the measured analyte data, statistics derived from the measured analyte data, etc.

Combining features determined from measured analyte data with a model that is insensitive to sensor variabilities advantageously increases the accuracy and reliability of the disease prediction, and eliminates many of the inconsistencies and disadvantages of conventional tests.

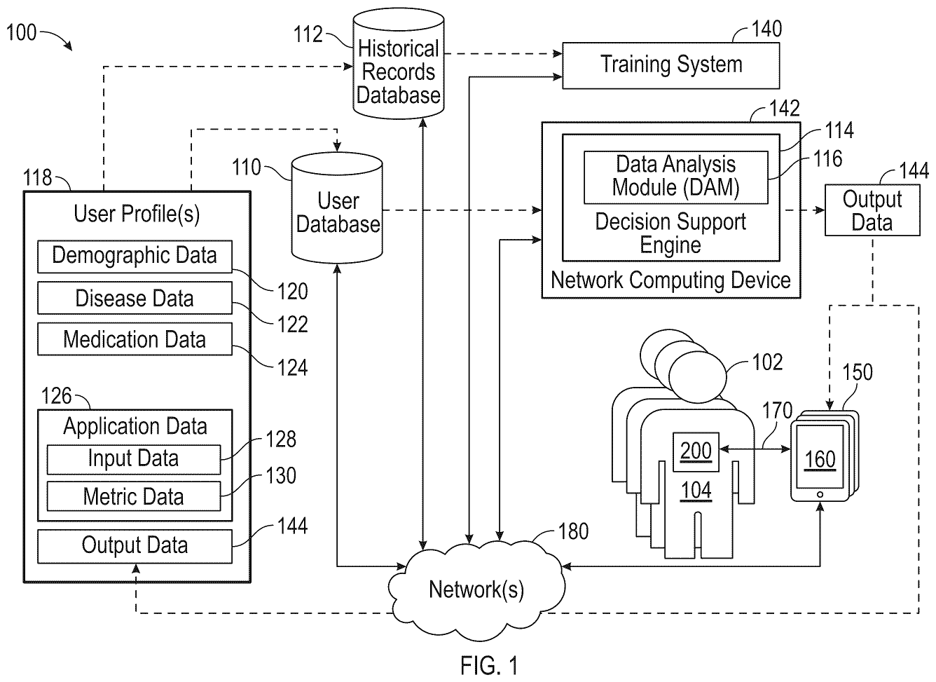

FIG. 1 illustrates aspects of health management system 100, in accordance with embodiments of the present disclosure.

Generally, health management system 100 provides disease predictions for each user 102 based on measured analyte data acquired by CAM system 200 worn by each user 102. In some embodiments, health management system 100 may also provide treatment recommendations for each user 102 in addition to disease predictions.

In certain embodiments, health management system 100 includes, inter alia, user database 110, historical records database 112, training system 140 connected to network(s) 180, network computing device 142 connected to network(s) 180, mobile computing devices (or display devices) 150 connected to network(s) 180, and CAM systems 200. Network(s) 180 may include one or more local area networks (LANs), wireless LANs (WLANs), low power wide area networks (LPWANs), wide area networks (WANs), cellular networks (such as 3G, 4G, LTE, 5G, 6G, etc.), the Internet, etc., employing various network topologies and protocols (hereinafter “network 180”). For example, network 180 may also include various combinations of wired and/or wireless physical layers, such as, for example, copper wire or coaxial cable networks, fiber optic networks, WiFi networks, Bluetooth mesh networks, CDMA, FDMA and TDMA cellular networks, etc.

User database 110 may be hosted by a network database server connected to network 180; alternatively, user database 110 may be hosted by network computing device 142 (indicated by a dashed line). User database 110 may store user profile 118 for each user 102 which may include, inter alia, demographic data 120, disease data 122, medication data 124, application data 126 including input data 128 (such as measured analyte data) and metric data 130, and output data 144 (such as a disease prediction). Similarly, historical records database 112 may be hosted by a network database server connected to network 180; alternatively, historical records database 112 may be hosted by training system 140 (indicated by a dashed line). Historical records database 112 may store, inter alia, historical analyte data and historical outcome data associated with the historical analyte data. The historical outcome data may include, inter alia, clinical disease diagnoses that indicate whether each user of the population has been clinically diagnosed with the particular disease based on one or more independent sources, such as sources that did not consider the historical analyte data.

Training system 140 is configured to evaluate, select and train disease prediction models in accordance with embodiments of the present disclosure. Training system 140 may include one or more network computing devices.

Network computing device 142 is configured to store and execute decision support engine (DSE) 114, as well as other software modules, applications, etc., to perform certain functionality described below. DSE 114 may include, inter alia, data analysis module (DAM) 116, as well as other software modules. In certain embodiments, training system 140 may include network computing device 142.

Display devices 150 are configured to store and execute one or more software applications that present one or more GUIs 160 to display certain data including, inter alia, input data 128 (such as measured analyte data, etc.), output data 144 (such as disease predictions, etc.), etc. In certain embodiments, at least a portion of DSE 114 and DAM 116 may be stored and executed by display device 150.

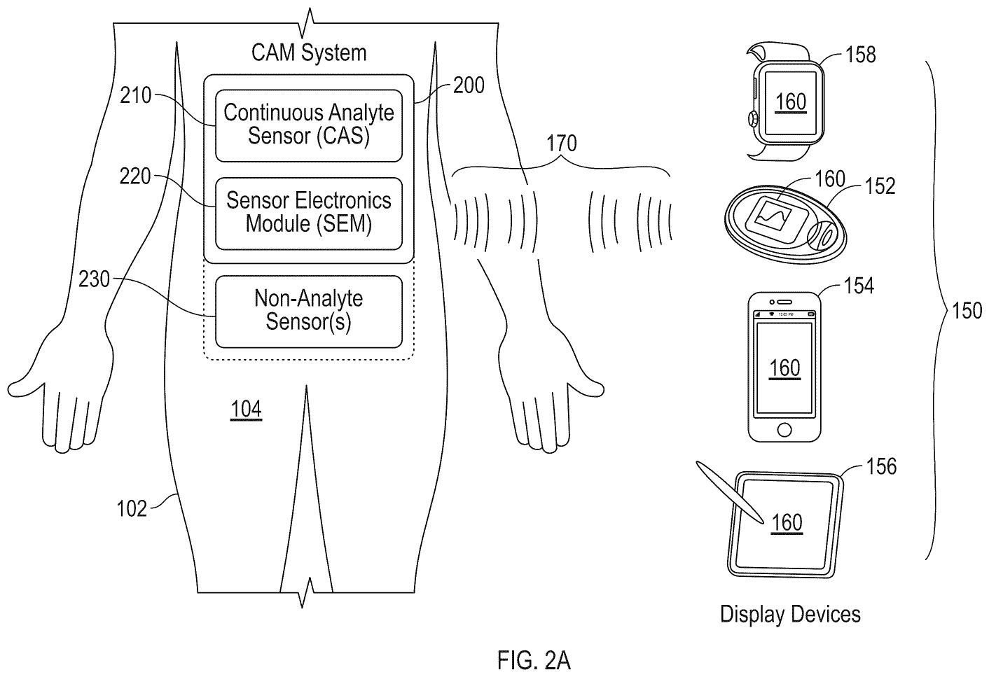

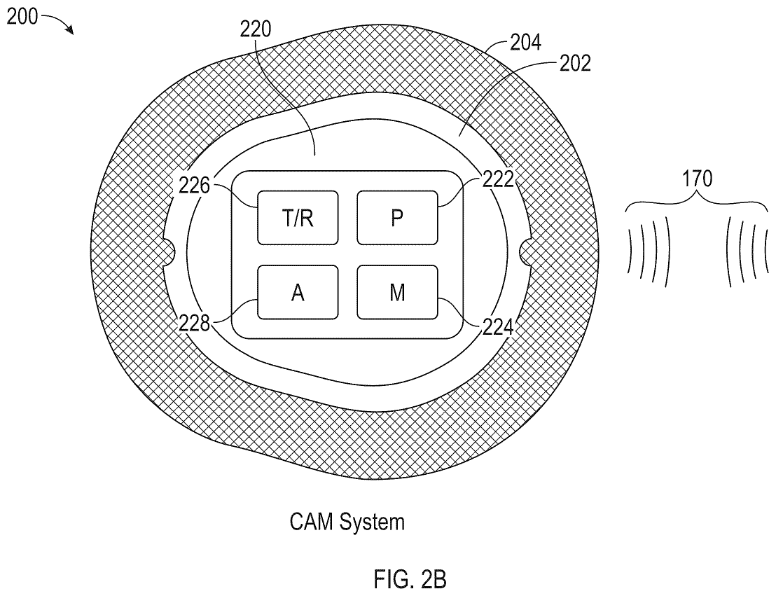

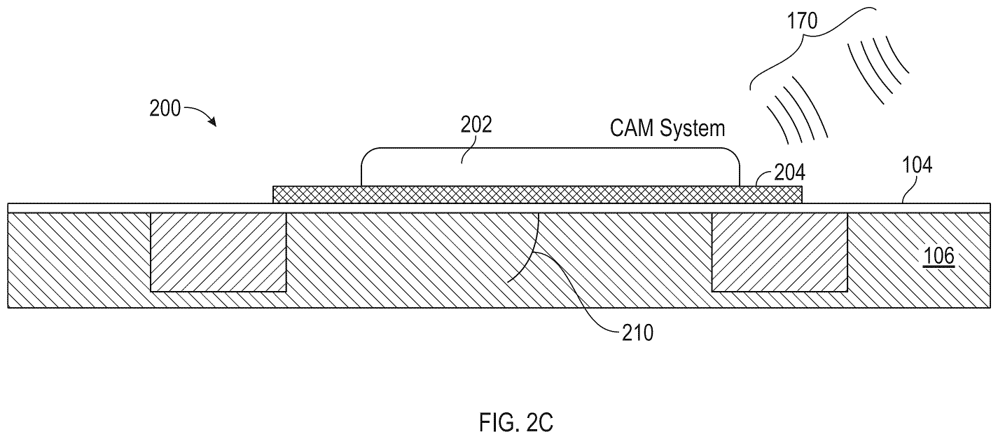

CAM systems 200 are configured to operate continuously to monitor one or more analytes for users 102. Each CAM system 200 is worn by a user 102, and may be coupled to a display device 150 via wireless connection 170 to transfer measured analyte data (and other data) to display device 150. Wireless connection 170 may be a Bluetooth connection, a Bluetooth Low Energy (BLE) connection, an RFID or NFC connection, an IEEE 802.11 connection (Wi-Fi), etc. CAM system 200 is described in more detail with respect to FIGS. 2A, 2B, 2C.

The term “analyte” as used herein is a broad term used in its ordinary sense, including, without limitation, to refer to a chemical substance, compound, molecule, clement, etc., in a biological fluid (such as blood, interstitial fluid, cerebral spinal fluid, lymph fluid, urine, etc.) that may be identified or measured, and analyzed.

Analytes may include naturally occurring substances, artificial substances, pharmacologic agents, metabolites, ions, blood gasses, hormones, neurotransmitters, vitamins, minerals, peptides, pathogens, toxins, and/or reaction products. Analytes for measurement by the devices and methods of the present disclosure may include (but may not be limited to) glucose; lactate; potassium; troponin; creatinine; ketone; acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgoninc (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; creatinine phosphokinase (CPK); cyclosporin A; cystatin C; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sisomicin; somatomedin C; specific antibodies recognizing any one or more of the following that may include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas acruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urca; uroporphyrinogen I synthase; vitamin A; white blood cells; and zinc protoporphyrin. Salts, sugar, protein, fat, vitamins, and hormones naturally occurring in blood or interstitial fluids may also constitute analytes in certain implementations. Ions are a charged atoms or compounds that may include the following (sodium, potassium, calcium, chloride, nitrogen, or bicarbonate, for example). The analyte may be naturally present in the biological fluid, for example, a metabolic product, a hormone, an antigen, an antibody, an ion etc. Alternatively, the analyte may be introduced into the body or exogenous, for example, a contrast agent for imaging, a radioisotope, a chemical agent, a fluorocarbon-based synthetic blood, a challenge agent analyte (such as introduced for the purpose of measuring the increase and or decrease in rate of change in concentration of the challenge agent analyte or other analytes in response to the introduced challenge agent analyte), or a drug or pharmaceutical composition, including but not limited to exogenous insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine The metabolic products of drugs and pharmaceutical compositions are also contemplated analytes. Analytes such as neurochemicals and other chemicals generated within the body may also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

In certain embodiments, CAM system 200 is configured to continuously measure one or more analytes and transmit the measured analyte data to an electric medical records (EMR) system (not shown in FIG. 1). An EMR system includes one or more network computing devices that host a software platform that is configured to receive, store and manage medical data. An EMR system is generally used throughout hospitals and/or other caregiver facilities to document clinical information on patients over long periods. EMR systems organize and present data in ways that assist clinicians with, for example, interpreting health conditions and providing ongoing care, scheduling, billing, and follow up. Data contained in an EMR system may also be used to create reports for clinical care and/or disease management for a patient. In certain embodiments, the EMR system may be in communication with network computing device 142 over network 180 to perform certain techniques described herein. In other embodiments, an EMR system may provide access to population-level health statistics, health economics, and the generation of clinical evidence or assessment of healthcare outcomes. In particular, as described herein, DSE 114 may access the EMR system to obtain data associated with a user 102, such as measured analyte data, for disease prediction purposes. In some cases, DSE 114 may provide the disease prediction to the EMR system.

CAM system 200 is configured to continuously measure one or more analyte concentration levels, and then transmit measured analyte data to display device 150 over wireless connection 170. In certain embodiments, a single-analyte sensor may be configured to generate an analog sensor signal that is proportional to the concentration level of a respective analyte, and a sensor electronics module may be configured to sample the analog sensor signal, generate measured analyte data, and transmit the measured analyte data to a display device 150. In certain embodiments, CAM system 200 periodically transmits the measured analyte data to display device 150 during the wear session. In other embodiments, CAM system 200 stores the measured analyte data in a memory, and transmits the measured analyte data to display device 150 at the conclusion of the wear session.

In certain embodiments, CAM system 200 may include multiple single-analyte sensors, and each single-analyte sensor generates an analog sensor signal that is proportional to the concentration level of a particular analyte. In other embodiments, CAM system 200 may include a multi-analyte sensor that generates multiple analog sensor signals, and cach analog sensor signal is proportional to the concentration level of a particular analyte. In further embodiments, CAM system 200 may include multiple multi-analyte sensors, a combination of single-analyte sensors and multi-analyte sensors, etc.

In certain embodiments, CAM system 200 may transmit the measured analyte data directly to network computing device 142 via network 180 for review, retrieval, execution of further analytics, etc. In such embodiments, CAM system 200 may be equipped with a mobile internet of things (IoT) interface, such as an LPWAN transceiver (such as LTE-M, Cat-M1, NB-IoT, etc.), a cellular radio transceiver, a Wi-Fi transceiver, etc., to transmit the measured analyte data over network 180.

Display devices 150 may be mobile computing devices that are wirelessly connected to network 180, using a WLAN, a cellular network, etc. In certain embodiments, display devices 150 may include a CAM data receiver, a smartphone, a tablet computer, a smartwatch, a laptop computer, etc. In some embodiments, display device 150 may transmit the measured analyte data to one or more other individuals having an interest in the health of the patient (such as a family member or physician for real-time treatment and care of the patient).

Generally, display device 150 is configured to receive and process measured analyte data from CAM system 200, and may store and execute one or more applications, such as a mobile health application, etc. In particular, display device 150 may store information about a user, including the user's measured analyte data, in a user profile 118 that is associated with the user. These data may be stored by display device 150 as well as user database 110.

Generally, DSE 114 may include one or more software modules, such as DAM 116, etc. In certain embodiments, DSE 114 may be stored and executed by network computing device 142, which communicates with display device 150 over network 180. In other embodiments, the software modules (or relevant functionality) may be distributed across multiple devices, and a portion of DSE 114 may be stored and executed by display device 150 and/or CAM system 200, while the remaining portion of DSE 114 may be stored and executed by network computing device 142. In some other embodiments, DSE 114 may be stored and executed by display device 150 and/or CAM system 200. Generally, DSE 114 may provide disease predictions based on the measured analyte data. In certain embodiments, DSE 114 may provide decision support recommendations based on information included in user profile 118.

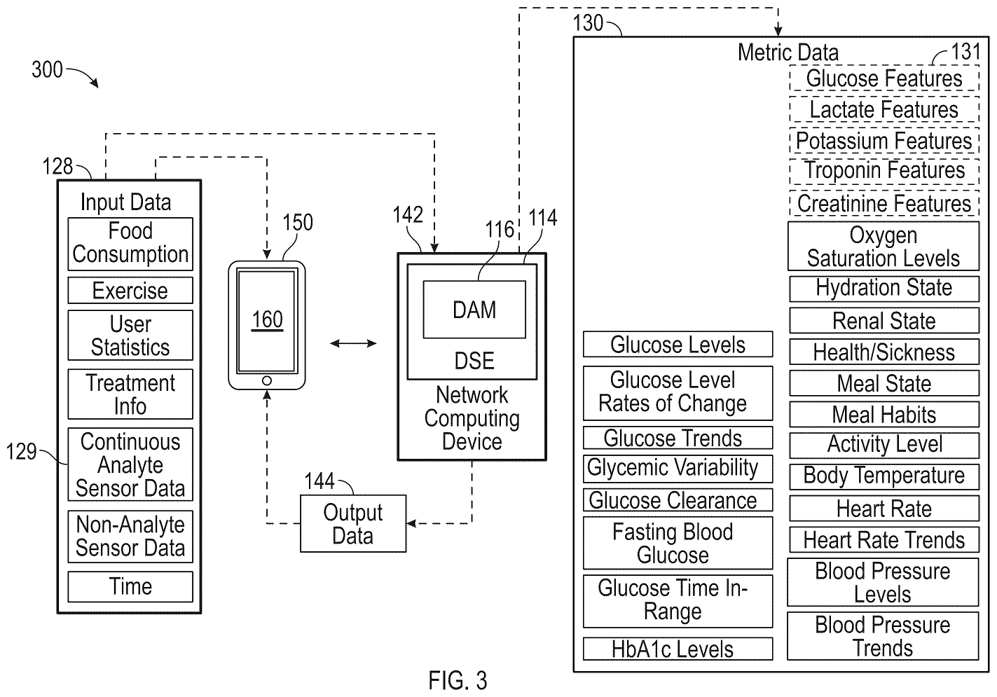

User profile 118 may include information collected about the user. For example, display device 150 may collect and store input data 128, including the measured analyte data received from CAM system 200, in user profile 118. In certain embodiments, input data 128 may include other data in addition to measured analyte data received from CAM system 200. For example, additional input data 128 may be acquired through manual user input, one or more other non-analyte sensors or devices, various processes executing on display device 150, etc. Input data 128 of user profile 118 are described in further detail below with respect to FIG. 3.

DAM 116 may be configured to generate metric data 130 based on input data 128. Metric data 130, discussed in more detail below with respect to FIG. 3, are generally indicative of the health or state of a user, such as one or more of the user's physiological state, trends associated with the health or state of a user, analyte features, etc. In certain embodiments, DSE 114 may provide disease prediction, guidance, etc., to a user based on metric data 130. As shown, metric data 130 are also stored in user profile 118.

User profile 118 also includes demographic data 120, disease data 122, and/or medication data 124 (such as type of medication, brand of medication, dosage, frequency of administration). In certain embodiments, such information may be provided through user input or obtained from certain data sources (such as electronic medical records, EMR systems, etc.). In certain embodiments, demographic data 120 may include one or more of the user's age, body mass index (BMI), ethnicity, gender, etc. In certain embodiments, disease data 122 may include information about a condition of a user, such as whether the user has been previously diagnosed with or experienced various diseases, such as diabetes, liver disease, kidney disease, heart disease, hyperglycemia, hypoglycemia, co-morbidities, etc. In certain embodiments, information about a user's condition may also include the length of time since diagnosis, the level of control, level of compliance with condition management therapy, other types of diagnosis (such as heart disease, obesity) or measures of health (such as heart rate, exercise, stress, sleep, etc.), and/or the like.

In certain embodiments, medication data 124 may include information about the amount, frequency, and type of a medication taken by a user. In certain embodiments, the amount, frequency, and type of a medication taken by a user is time-stamped and correlated with the user's analyte levels, thereby, indicating the impact the amount, frequency, and type of the medication had on the user's analyte levels. In certain embodiments, medication data 124 may include information about the prescribed dosage/frequency and the consumption of one or more inhibitors that may be prescribed to a patient for the purpose of treating a disease. For example, user 102 may be prescribed inhibitors (such as sodium glucose cotransporter 2 or SGLT2) to help control blood glucose levels by blocking absorption of glucose by the body.

As described in more detail below, health management system 100 may be configured to determine an inhibitor effectiveness or an optimal inhibitor dosage and frequency to be prescribed to different users based on medication data 124. In particular, health management system 100 may be configured to identify one or more optimal prescriptions based on the health of the patient when one or more medications are prescribed, as well as the condition(s) of the patient to be treated.

In certain embodiments, medication data 124 may include information about consumption of other drugs for the control of blood glucose. For example, medication data 124 may include metformin, thiazolidinediones, sulfonylureas, GLP-1 receptor agonists, glucagon, and/or insulin dosage and frequency. The glucose information may include information manually provided by the user and/or information provided by an automated insulin delivery (AID) device.

In certain embodiments, user profile 118 may be dynamic because at least part of the information that is stored in user profile 118 may be revised over time and/or new information may be added to user profile 118 by DSE 114, display device 150, etc. Accordingly, information in user profile 118 stored in user database 110 may provide an up-to-date repository of information related to a user.

User database 110 may be implemented as any type of data store, such as relational databases, non-relational databases, key-value data stores, file systems including hierarchical file systems, etc. In some embodiments, user database 110 may be distributed. For example, user database 110 may comprise persistent storage devices, which are distributed. Furthermore, user database 110 may be replicated so that the storage devices are geographically dispersed.

Similarly, historical records database 112 may be implemented as any type of data store, such as relational databases, non-relational databases, key-value data stores, file systems including hierarchical file systems, etc. In some embodiments, historical records database 112 may be distributed. For example, historical records database 112 may comprise persistent storage devices, which are distributed. Furthermore, historical records database 112 may be replicated so that the storage devices are geographically dispersed.

Although depicted as separate databases for conceptual clarity, in some embodiments, user database 110 and historical records database 112 may be combined into a single database. In other words, the historical and current data related to users of CAM system 200, as well as historical data related to patients that were not previously users of CAM system 200, may be stored in a single database.

User database 110 may include user profiles 118 associated with a number of users who similarly interact with respective display devices 150. User profiles 118 stored in user database 110 may be accessible over network 180. As described above, DSE 114, and more specifically DAM 116, may fetch input data 128 from user database 110 and generate metric data 130 which may then be stored as application data 126 in user profile 118.

In certain embodiments, user profiles 118 stored in user database 110 may also be stored in historical records database 112. User profiles 118 stored in historical records database 112 may provide a repository of up-to-date information and historical information for each user. Thus, historical records database 112 essentially provides all data related to each user of CAM system 200. In certain embodiments, the data may be stored with an associated timestamp to identify when information related to a user has been obtained, updated, etc.

Further, historical records database 112 may maintain time series data collected for users over a period of time (such as 5 years), including for users who use CAM system 200. Further, in certain embodiments, historical records database 112 may also include data for one or more patients who are not users of CAM system 200. For example, historical records database 112 may include information (such as user profiles) related to one or more patients treated by a healthcare physician. Data stored in historical records database 112 may be referred to herein as population data.

Data related to each patient stored in historical records database 112 may provide time series data collected over a disease lifetime of the patient. For example, the data may include information about the patient prior to being diagnosed and information associated with the patient during the lifetime of the treatment, including information related to level of treatment required, as well as information related to other diseases or conditions. Such information may indicate symptoms of the patient, physiological states of the patient, measured analyte data for the patient, states/conditions of one or more organs of the patient, habits of the patient (such as activity levels, food consumption, etc.), medication prescribed, etc., throughout the lifetime of the treatment.

In certain embodiments, DSE 114 may include one or more trained models configured to predict disease for a user 102 based on information provided by CAM system 200. For example, DSE 114 may include one or more trained ML models provided by training system 140. In some embodiments, training system 140 may store and execute DSE 114. That is, the model may be trained and then hosted by training system 140.

Generally, training system 140 is configured to develop disease prediction models. As discussed above, training system 140 adds analyte sensor bias to the historical analyte data stored within historical records database 112 to generate biased analyte data, and associate the biased analyte data with the respective historical outcome data. Training system 140 then extracts features from the biased analyte data, and evaluates one or more models based on different combinations of features. More particularly, for each model under evaluation, training system 140 generates disease predictions based on different combinations of features extracted from the biased analyte data, and evaluates the disease predictions based on the historical outcome data associated with the biased analyte data. Training system 140 then selects the model and combination of features that produces the best prediction performance with respect to the biased analyte data based on the performance metric and the robustness metric. Training system 140 then trains the selected model based on the selected combination of features determined from the historical analyte data, and the clinical disease diagnoses associated with the historical analyte data.

Training system 140 may also provide the trained models to DSE 114 for disease prediction. For example, DSE 114 may obtain user profile 118 associated with a user, provide certain information contained therein to a trained model, and then output a disease prediction, such as shown as output data 144 in FIG. 1.

Generally, the disease prediction indicates the absence of the disease or the presence of the disease in real-time or within a certain time frame. Output data 144 may be stored in user database 110, provided to the user 102 through GUI 160 presented on display device 150, provided to the user's caretaker (such as a parent, a relative, a guardian, a teacher, a nurse, etc.), provided to the user's physician, or any other individual that has an interest in the wellbeing of the user for purposes of improving the user's health, such as, in some cases by effectuating the recommended treatment.

In certain embodiments, output data 144 may be stored in user profile 118. In certain embodiments, output data 144 may include a disease prediction, one or more treatment recommendations based on the disease prediction, treatment efficacy, identification of one or more disease indicators, etc. For example, in certain embodiments, output data 144 may include a disease prediction for a user 102, a treatment recommendation for an update in medication, medication dosage, medication frequency of use, etc. In certain embodiments, output data 144 may be a prediction as to the risk of the onset of one or more diseases. In certain embodiments, output data 144 may include a prediction as to the risk of a user having hyperglycemia and/or hypoglycemia, pre-diabetes, type 2 diabetes, gestational diabetes, etc. In certain embodiments, output data 144 may include a prediction as to a mortality risk of the patient. In certain embodiments, output data 144 may include patient-specific treatment decisions or recommendations for glucose control for the patient. In certain embodiments, output data 144 may include a recommendation relating to the use of an inhibitor, a recommendation relating to the user of insulin, etc.

In certain embodiments, output data 144 may be stored in user database 110 and continuously updated by DSE 114. Accordingly, previous diagnoses and/or physiological metrics of the user, originally stored as output data 144 in user profile 118 in user database 110 and then passed to historical records database 112, may provide an indication of the effectiveness of the current treatment or may provide a likelihood of onset of the predicted disease in a user in a given time period.

In certain embodiments, a user's own historical data may be used to provide decision support and insight around the user's physiological condition and/or condition onset. For example, a user's historical data may be used as a baseline to indicate improvements or deterioration in the user's condition. As an illustrative example, a user's data from two weeks prior may be used as a baseline that may be compared with the user's current data to identify an improvement or deterioration in glucose levels of the user and, thereby, a whether the risk associated with a future hyperglycemic or hypoglycemic event has increased or decreased.

FIG. 2A depicts a diagram of CAM system 200 and display devices 150, in accordance with embodiments of the present disclosure.

In certain embodiments, CAM system 200 includes, inter alia, continuous analyte sensor (CAS) 210, sensor electronic module (SEM) 220, and a power source, such as a battery. One or more non-analyte sensors (NAS) 230 or other devices may also be coupled to SEM 220.

Generally, CAS 210 may include one or more single-analyte sensors, one or more multi-analyte sensors, a combination of single-analyte sensors and multi-analyte sensors, etc. Each single-analyte sensor generates an analog sensor signal that is proportional to the concentration level of a particular analyte. Similarly, each multi-analyte sensor generates multiple analog sensor signals, and each analog signal is proportional to the concentration level of a particular analyte. As an illustrative example, CAS 210 may include a single-analyte sensor configured to measure glucose concentration levels, and one or more multi-analyte sensors configured to measure lactate concentration levels, potassium concentration levels, troponin concentration levels, creatinine concentration levels, etc. As another illustrative example, CAS 210 may include a multi-analyte sensor configured to measure glucose concentration levels, lactate concentration levels, potassium concentration levels, troponin concentration levels, creatinine concentration levels, etc.

Accordingly, CAS 210 is configured to generate at least one analog sensor signal that is proportional to the concentration level of particular analyte, and SEM 220 is configured to sample the analog sensor signal, generate measured analyte data, and transmit the measured analyte data to display device 150 via wireless connection 170. SEM 220 is configured to sample the analog sensor signal at a particular sampling period (or rate), such as every 1 second (1 Hz), 5 seconds, 10 seconds, 30 seconds, 1 minute, 3 minutes, 5 minutes, 10 minutes, 30 minutes, etc., and to transmit the measured analyte data to display device 150 at a particular transmission period (or rate), which may be the same as (or longer than) the sampling period, such as every 1 minute (0.016 Hz), 5 minutes, 10 minutes, 30 minutes, 60 minutes, etc., at the conclusion of the wear period, etc. Depending on the sampling and transmission periods, the measured analyte data transmitted to display device 150 include at least one analyte concentration level measurement having an associated time tag, sequence number, etc.

CAS 210 may be a non-invasive device, a subcutaneous device, a transcutaneous device, a transdermal device, a dermal device, an intradermal device, a subdermal device, an intravascular device, etc. In certain embodiments, CAS 210 may be configured to continuously measure analyte concentration levels using one or more measurement techniques, such as enzymatic, immunometric, aptameric, amperometric, voltametric, potentiometric, impedimetric, conductimetric, chemical, physical, electrochemical, spectrophotometric, polarimetric, calorimetric, iontophoretic, radiometric, immunochemical, optical, ion-selective, etc.

Display devices 150 may be mobile computing devices that are connected network 180. In certain embodiments, display devices 150 may include CAM data receiver 152, smartphone 154, tablet computer 156, smartwatch 158, laptop computer (not shown), etc. In some embodiments, display devices 150 may be non-mobile computing devices (such as a desktop computer, etc.) that are connected to network 180.

In certain embodiments, display devices 150 are configured for displaying data, including measured analyte data, which may be transmitted by SEM 220. Display devices 150 may include a touchscreen display for displaying data to a user and receiving inputs from the user. For example, GUI 160 may be presented to the user for such purposes. In some embodiments, display devices 150 may include other types of user interfaces such as a voice user interface instead of, or in addition to, a touchscreen display for communicating data to the user of display device 150 and receiving user inputs.

In some embodiments, one, some, or all of display devices 150 are configured to display or otherwise communicate the data as it is communicated from SEM 220 (such as in a data package that is transmitted to respective display devices 150), without any additional prospective processing required for calibration and real-time display of the data. In certain embodiments, the display devices 150 may be configured for providing alerts/alarms/notifications based on the displayable data.

For example, CAM data receiver 152 may be a custom display device specially designed for displaying certain types of data associated with measured analyte data received from SEM 220. For another example, smartphone 154 may use a commercially available operating system (OS), and may be configured to display a graphical representation of the continuous measured analyte data (such as including current and historic data) using GUI 160.

Because different display devices 150 provide different user interfaces, the content of the data packages (such as amount, format, and/or type of data to be displayed, alarms, etc.) may be customized (such as programmed differently by the manufacture and/or by an end user) for each particular display device 150. Accordingly, in certain embodiments, a number of different display devices 150 may be in direct wireless communication with a SEM 220 of a CAM system 200 worn by a user 102 during a wear session to enable a number of different types and/or levels of display and/or functionality associated with the displayable data. In certain embodiments, the type of alarms customized for each particular display device 150, the number of alarms customized for each particular display device 150, the timing of alarms customized for each particular display device 150, and/or the threshold levels configured for each of the alarms (such as for triggering) are based on output data 144.

NAS 230 may include a temperature sensor, an altimeter sensor, an accelerometer sensor, a respiration rate sensor, a sweat sensor, a heart rate sensor, an electrocardiogram (ECG) sensor, a blood pressure sensor, a respiratory sensor, an oxygenated hemoglobin sensor (spO2), etc. Other devices may be coupled to SEM 220, such as an insulin pump, a peritoneal dialysis machine, a hemodialysis machine, etc.

FIGS. 2B, 2C depict top and side views of CAM system 200, respectively, in accordance with embodiments of the present disclosure.

CAM system 200 includes housing 202 enclosing SEM 220, and adhesive pad 204 disposed on the bottom surface of housing 202. CAS 210 protrudes from the bottom surface of housing 202 and adhesive pad 204. CAM system 200 is configured to be worn on epidermis 104 of user 102 at a convenient location, such as the back of the upper arm, the abdomen, etc.

CAM system 200 may be battery powered, and, in certain embodiments, the battery may be replaced or recharged if necessary. SEM 220 is coupled to CAS 210, and includes electronic circuitry configured to acquire, process, store and transmit measured analyte data, as well as other information, to display devices 150 for presentation to user 102.

In certain embodiments, CAS 210 may be a single-analyte sensor that includes a percutaneous wire that has a proximal portion coupled to SEM 220 and a distal portion with several electrodes. A measurement (or working) electrode may be coated, covered, treated, embedded, etc., with one or more chemical molecules that react with a particular analyte, and a reference electrode may provide a reference electrical voltage. The measurement electrode may generate the analog sensor signal, which is conveyed along a conductor that extends from the measurement electrode to the proximal portion of the percutaneous wire that is coupled to SEM 220. After CAM system 200 has been applied to epidermis 104 of user 102, CAS 210 penetrates epidermis 104, and the distal portion extends into the dermis and/or subcutaneous tissue 106 under epidermis 104 (as depicted in FIG. 2B). Other configurations of CAS 210 may also be used, such as a multi-analyte sensor that includes multiple measurement electrodes, each generating an analog sensor signal that represents the concentration levels of a particular analyte.

In certain embodiments, CAS 210 may incorporate a thermocouple within, or alongside, the percutaneous wire to provide an analog temperature signal to SEM 220, which may be used to correct the analog sensor signal or the measured analyte data for temperature. In other embodiments, the thermocouple may be incorporated into SEM 220 above adhesive pad 204, or, alternatively, the thermocouple may contact epidermis 104 of user 102 through openings in adhesive pad 204.

In certain embodiments, SEM 220 includes, inter alia, processor (P) 222, memory (M) 224, transceiver or transmitter/receiver (T/R) 226, one or more antennae (A) 228 coupled to transceiver 226, analog signal processing circuitry, analog-to-digital (A/D) signal processing circuitry, digital signal processing circuitry, a power source for CAS 210 (such as a potentiostat), ctc.

Processor 222 may be a general-purpose or application-specific microprocessor, an application-specific integrated circuit (ASIC), a field-programmable gate array (FPGA), etc., that executes instructions to perform control, computation, input/output, etc. functions for CAM system 200. Processor 222 may include a single integrated circuit, such as a micro-processing device, or multiple integrated circuit devices and/or circuit boards working in cooperation to accomplish the appropriate functionality. In certain embodiments, processor 222, memory 224, transmitter/receiver 226, the A/D signal processing circuitry, and the digital signal processing circuitry may be combined into a system-on-chip (SoC).

In operation, CAS 210 and adhesive pad 204 may be assembled to form an application assembly, where the application assembly is configured to be applied to the user's epidermis 104 so that CAS 210 is subcutaneously inserted as depicted. In such scenarios, SEM 220 may be attached to the assembly after application to the user's epidermis 104 via an attachment mechanism (not shown). Alternatively, SEM 220 may be incorporated as part of the application assembly, such that CAS 210, adhesive pad 204 and SEM 220 can all be applied at once to the user's epidermis 104. In one or more embodiments, this application assembly is applied to the user's epidermis 104 using a separate sensor applicator (not shown).

Unlike the fingersticks required by certain conventional analyte measurement techniques, for example, user-initiated application of CAM system 200 with a sensor applicator is nearly painless and does not require the withdrawal of blood. Moreover, the automatic sensor applicator generally enables the user to embed CAS 210 subcutaneously into the user's epidermis 104 without the assistance of a clinician or health care provider.

CAM system 200 may be removed by peeling adhesive pad 204 from the user's epidermis 104. It is to be appreciated that CAM system 200 and its various components are illustrated as one example form factor, and CAM system 200 and its components may have different form factors without departing from the spirit or scope of the described techniques.

Generally, processor 222 is configured to sample the analog sensor signal using the A/D signal processing circuitry at regular intervals (such as the sampling period), generate measured analyte data from the sampled analog sensor signal, and generate sensor data packages that include, inter alia, the measured analyte data. Processor 222 may store the measured analyte data in memory 224, and generate the sensor data packages at regular intervals (such as the transmission period) for transmission by T/R 226 to display device 150. Processor 222 may also add additional data to the sensor data packages, such as supplemental sensor information that includes a sensor identifier, a sensor status, temperatures that correspond to the measured analyte data, etc.

With respect to the supplemental sensor information, the sensor identifier represents information that uniquely identifies CAS 210 from other sensors, such as other sensors of other analyte monitoring devices, other sensors implanted previously or subsequently in the user's epidermis 104, and so on. By uniquely identifying CAS 210, the sensor identifier may also be used to identify other aspects about CAS 210, such as a manufacturing lot of CAS 210, packaging details of CAS 210, shipping details of CAS 210, and so on. In this way, various issues detected for sensors manufactured, packaged, and/or shipped in a similar manner as CAS 210 may be identified and used in different ways in order to calibrate the measured analyte data, to notify users of defective sensors, to notify manufacturing facilities of machining issues, and so forth.

The sensor status of the supplemental sensor information represents a state of CAS 210 at a given time, such as a state of the sensor at a same time one of the measured analyte data is produced. To this end, the sensor status may include an entry for each of the measured analyte data, such that there is a one-to-one relationship between the measured analyte data and statuses captured in the supplemental sensor information. For example, the sensor status may describe an operational state of CAS 210. In certain embodiments, processor 222 may identify one of a number of predetermined operational states for a given measurement. The identified operational state may be based on the communications from CAS 210 and/or characteristics of those communications.

In certain embodiments, a lookup table, stored in memory 224, may include the predetermined number of operational states and bases for selecting one state from another. For example, the predetermined states may include a “normal” operation state where the bases for selecting this state may include an analog sensor signal from CAS 210 that falls within thresholds indicative of normal operation, an analog temperature signal that is within a threshold of suitable temperatures to continue operation as expected, etc. The predetermined states may also include operational states that indicate that one or more characteristics of the analog sensor signal from CAS 210 are outside of normal activity and may result in potential errors in the measured analyte data, such as an analog sensor signal from CAS 210 that is outside a threshold of expected signal strength, an environmental temperature that is outside suitable temperatures to continue operation as expected, detecting that the user 102 has physically rolled onto CAM system 200, etc.

FIG. 3 illustrates input data 128 and metric data 130 for use by health management system 100, in accordance with embodiments of the present disclosure.

More particularly, FIG. 3 illustrates input data 128 on the left, display device 150 and network computing device 142 in the middle, and metric data 130 on the right. Generally, display device 150 stores and executes one or more related applications and presents GUI 160 to the user, while network computing device 142 stores and executes DSE 114 (including DAM 116), as well as other applications. As described above, in certain embodiments, a portion of DSE 114 may be stored and executed by display device 150 (or CAM system 200), while the remaining portion of DSE 114 may be stored and executed by network computing device 142. In other embodiments, DSE 114 may be stored and executed by display device 150 (or CAM system 200).

In certain embodiments, metric data 130 includes various types of data, such as discrete numerical values, ranges, qualitative values (high/medium/low, stable/unstable, rate of change, points of inflection, etc.), etc. Display device 150 obtains input data 128 through one or more channels such as manual user input, sensors/monitors, other applications executing on display device 150, EMR systems, etc.). As mentioned above, in certain embodiments, DSE 114 (including DAM 116) may process input data 128 to generate metric data 130, and generate a discase prediction based on certain elements of metric data 130.

For example, DSE 114 may process continuous analyte sensor data 129, such as measured glucose data provided by CAM system 200, to determine glucose features 131, and then generate a diabetes prediction as output data 144, such as diabetes or not diabetes, based on a combination of glucose features 131. In this example, training system 140 has evaluated, selected and trained a model to predict diabetes based on a combination of glucose features that have been extracted from historical glucose data, and DSE 114 executes the model to generate the diabetes prediction. The combination of glucose features 131 and the combination of glucose features extracted from the historical analyte data include the same type of features.

In certain embodiments, starting with input data 128, food consumption information may include information about one or more of meals, snacks, and/or beverages, such as one or more of the size, content (milligrams (mg) of sodium, potassium, carbohydrate, fat, protein, etc.), sequence of consumption, and time of consumption. In certain embodiments, food consumption may be provided by a user through manual entry, by providing a photograph through an application that is configured to recognize food types and quantities, by scanning a bar code or menu, and/or interrogating an NFC/RFID tag. In various examples, meal size may be manually entered as one or more of calories, quantity (such as “three cookies”), menu items (such as “Royale with Cheese”), and/or food exchanges (such as 1 fruit, 1 dairy). In some examples, meal information may be received by the related application(s) executing on display device 150. In some examples, meal information may be provided via one or more other applications synchronized with the related application(s), such as one or more other mobile health applications executed by display device 150. In such examples, the synchronized applications may include, such as an electronic food diary application, photograph application, etc.

In certain embodiments, food consumption information entered by a user may relate to nutrients consumed by the user. Consumption may include any natural or designed food or beverage. Food consumption information entered by a user may also be related to analytes, including any of the other analytes described herein.

In certain embodiments, exercise information may also be provided. Exercise information may be any information surrounding activities, such as activities requiring physical exertion by the user. For example, exercise information may range from information related to low intensity (such as walking a few steps) and high intensity (such as five mile run) physical exertion. In certain embodiments, exercise information may be provided, for example, by an accelerometer sensor or a heart rate monitor on a wearable device such as a watch, fitness tracker, and/or patch. In certain embodiments, exercise information may also be provided through manual user input and/or through a surrogate sensor and prediction algorithm measuring changes to heart rate (or other cardiac metrics). When predicting that a user is exercising based on his/her sensor data, the user may be asked to confirm if exercise is occurring, what type of exercise, and or the level of strenuous exertion being used during the exercise over a specific period. This data may be used to train the system to learn about the user's exercise patterns to reduce the need for confirmation questions as time progresses. Other analytes and sensor data may also be included in this training set, including analytes and other measured elements described herein including temporal elements such as time and day.

In certain embodiments, user statistics, such as one or more of age, height, weight, BMI, body composition (such as % body fat), stature, build, or other information may also be provided as an input. In certain embodiments, user statistics may be provided through GUI 160, by interfacing with an electronic source such as an electronic medical record, from measurement devices, etc. In certain embodiments, the measurement devices include one or more of a wireless, such as a Bluetooth-enabled, weight scale or camera, which may, for example, communicate with display device 150 to provide user data.

In certain embodiments, treatment information may also be provided as an input. Treatment information may include information about the type, dosage, and/or timing of when one or more medications (such as SGLT2, insulin) are to be taken by the user. As mentioned herein, the treatment information may include information about one or more inhibitors, one or more drugs known to reduce blood glucose levels, one or more drugs known to affect glucose, and/or one or more medications for treating one or more symptoms of acute or chronic conditions and diseases the user may have. The treatment information may include information regarding different lifestyle habits, surgical procedures, and/or other non-invasive procedures recommended by the user's physician. For example, the user's physician may recommend a user increase/decrease their carbohydrate intake, exercise for a minimum of thirty minutes a day, or increase an insulin dosage or other medication to maintain, improve, and/or reduce hyper-and/or hypoglycemic episodes, etc. As another example, a healthcare professional may recommend that a user engage in at-home treatment and/or treatment at a clinic. The treatment information may also indicate a patient's adherence to the prescribed type, dosage, and/or timing of medications. For example, the treatment/medication information may indicate whether and when exactly and with what dosage/type the medication was taken.

In certain embodiments, measured analyte data may include glucose concentration levels measured by at least a glucose sensor (or multi-analyte sensor configured to measure at least glucose) that is a part of CAM system 200. Glucose baselines, glucose level rates of change, glucose trends, glucose variability, glucose clearance, glucose time in-range, glucose features 131, etc., may also be determined from the measured glucose data acquired by CAM system 200. Additionally, fasting blood glucose and HbA1c levels may be provided as metric data 130.

Similarly, in certain embodiments, measured analyte data may include lactate concentration levels measured by at least a lactate sensor (or multi-analyte sensor configured to measure at least lactate) that is a part of CAM system 200, and lactate-related data may also be determined from the measured lactate data acquired by CAM system 200, such as lactate features.

In certain embodiments, measured analyte data may include potassium concentration levels measured by at least a potassium sensor (or multi-analyte sensor configured to measure at least potassium) that is a part of CAM system 200, and potassium-related data may also be determined from the measured potassium data acquired by CAM system 200, such as potassium features.

In certain embodiments, measured analyte data may include troponin concentration levels measured by at least a troponin sensor (or multi-analyte sensor configured to measure at least troponin) that is a part of CAM system 200, and troponin-related data may also be determined from the measured troponin data acquired by CAM system 200, such as troponin features.

In certain embodiments, measured analyte data may include creatinine concentration levels measured by at least a creatinine sensor (or multi-analyte sensor configured to measure at least creatinine) that is a part of CAM system 200, and creatinine-related data may also be determined from the measured creatinine data acquired by CAM system 200, such as creatinine features.

In certain embodiments, data may also be received from one or more non-analyte sensors 230. Data from non-analyte sensors 230 may include information related to a heart rate, heart rate variability (such as the variance in time between the beats of the heart), ECG data, a respiration rate, oxygen saturation, a blood pressure, or a body temperature (such as to detect illness, physical activity, etc.) of a user. In certain embodiments, electromagnetic sensors may also detect low-power radio frequency (RF) fields emitted from objects or tools touching or near the object, which may provide information about user activity or location.

In some embodiments, non-analyte sensors 230 may include a scanner/reader to detect medication related information (such as type, brand, dosage, frequency). Examples of a scanner may include a reader configured to detect near-field communication (NFC) and/or radio frequency identification (RFID) information provided by a corresponding active or passive tag provided with packaging or otherwise accompanying the medication. Another example of a scanner may be a barcode, QR, or other optical scanner capable of accessing information associated with a visual pattern provided on the packaging or otherwise associated with the medication.

In certain embodiments, data received from non-analyte sensors 230 may include data relating to a user's insulin delivery. In particular, data related to the user's insulin delivery may be received, via a wireless connection on a smart pen, via user input, and/or from an insulin pump. Insulin delivery information may include one or more of insulin manufacturer, insulin dosage, insulin formulation, insulin volume, basal vs bolus dose, intended pharmacokinetic profile (such as short-acting, long-acting), number of units of insulin delivered, time of delivery, etc. Other metrics, such as insulin action time or duration of insulin action, may also be received.

In certain embodiments, time may also be provided, such as time of day, UTC time or time from a real-time clock. Said real-time clock may be provided externally (synchronized to a server via a WiFi wireless connection) or may be embedded as an integrated circuit (RTC) within the wearable/sensor electronics. For example, measured analyte data may be timestamped to indicate a date and time when the analyte measurement was acquired by CAM system 200.

In certain embodiments, at least a portion of input data 128 may be acquired through GUI 160 of display device 150.

In certain embodiments, DAM 116 may determine, based on the measured analyte data and other data (such as GPS data), whether the user is engaging in an activity over a period of time that might affect the measured analyte data, such as engaging in exercise, consuming nutrients, etc. In certain embodiments, DAM 116 may first identify which measured analyte data are not to be used for calculating an analyte baseline by identifying which measured analyte data have been affected by an activity, such as consumption of food, exercise, medication, or other perturbation that would disrupt determination of the analyte baseline. DAM 116 may then exclude such measured analyte data when calculating the analyte baseline of a user. In other examples, DAM 116 may calculate the analyte baseline by first determining a percentage of the measured analyte data values during this time period that represent the lowest analyte values measured. DAM 116 may average these measured analyte data values to determine the analyte baseline.

In certain embodiments, an absolute maximum analyte concentration level may be determined from measured analyte data, health/sickness metrics, and/or other condition metrics. The absolute maximum analyte concentration level represents a user's maximum analyte concentration level determined to be safe over a period of time (such as hourly, weekly, daily, etc.). In certain embodiments, the absolute maximum analyte concentration level may be consistent across all users. In certain other embodiments, each patient may have a different absolute maximum analyte concentration level. In certain embodiments, absolute maximum analyte concentration level per patient may change over time. For example, a user may be initially assigned an absolute maximum analyte concentration level based on clinical data. This assigned absolute maximum analyte concentration level may be adjusted over time based on other sensor data, comorbidities, etc. for patient. The minimum analyte concentration level may be determined in a similar manner.

In certain embodiments, analyte thresholds other than an absolute maximum and/or minimum analyte concentration level of a user may be determined from measured analyte data, health/sickness metrics, other condition metrics, etc. Such analyte thresholds may represent maximum or minimum analyte concentration levels determined to be safe during certain activities, which may vary across different activities. For example, because exercise is known to affect certain analyte levels, maximum and/or minimum analyte thresholds for a user during exercise may be different than maximum and/or minimum analyte thresholds for user during other activities.

In certain embodiments, analyte concentration level rates of change may be determined from measured analyte data. For example, an analyte concentration level rate of change refers to a rate that indicates how one or more time-stamped measured analyte data values change in relation to one or more other time-stamped measured analyte data values. Analyte concentration level rates of change may be determined over one or more seconds, minutes, hours, days, etc.

In certain embodiments, determined analyte concentration level rates of change may be marked as “increasing rapidly” or “decreasing rapidly”. As used herein, “rapidly” may describe analyte concentration level rates of change that are clinically significant and pointing towards a trend of analyte concentration levels likely breaching absolute maximum analyte concentration level or absolute minimum analyte concentration level within a defined period of time. In other words, a predictive trend may, in some cases, indicate that a patient is likely to hit, for example, absolute maximum analyte concentration level within a specified time period (such as one or two hours) based on determined analyte concentration level rate of change. Accordingly, such an analyte concentration level rate of change may be marked as “increasing rapidly”. Similarly, a predictive trend may, in some cases, indicate that a patient is likely to hit absolute minimum analyte concentration level within a specified time period (such as one or two hours) based on analyte concentration level rate of change determined. Accordingly, such an analyte concentration level rate of change may be marked as “decreasing rapidly”.

In certain embodiments, analyte baseline rates of change may be determined from analyte baselines determined for a user over time.

In certain embodiments, an analyte clearance rate may be determined from measured analyte data following consumption of a known, or estimated, amount of analyte. The analyte clearance rates analyzed over time may be indicative of medication efficacy or onset of a condition. In particular, slope of a curve of analyte clearance during a first time period (such as after administration of an inhibitor) compared to slope of a curve of an analyte clearance during a second time period (such as after consuming same inhibitor) may be indicative of an effectiveness of a treatment.

In certain embodiments, analyte clearance rate may be determined by calculating a slope between a first value at to (such as during a period of increased analyte concentration levels) and the user's analyte baseline reached at 11. In certain embodiments, an analyte clearance rate may be calculated over time until increased analyte concentration levels of the user reach some value relative to user's analyte baseline (such as a percentage of a user's analyte baseline). Analyte clearance rates calculated over time may be time-stamped and stored in user's profile 118.

In certain embodiments, a standard deviation of analyte concentration levels may be determined from measure analyte data. In some examples, a standard deviation of one or more analyte concentration levels may be determined based on variability of one or more analyte concentration levels as compared to an average analyte concentration level over one or more time periods. In some embodiments, a time-in-range metric (not shown) may be determined from measured analyte data. For example, with an established upper limit and lower limit, time period during which measured analyte data was between upper and lower limits can be determined. time-in-range may be determined for individual instances of measured analyte data being in-range or may be determined over a predetermined length of time (one day) for which each individual in-range periods are summed.

In certain embodiments, analyte trends may be determined based on analyte concentration levels over certain periods of time. In certain embodiments, analyte trends may be determined based on analyte baselines over certain periods of time. In certain embodiments, analyte trends may be determined based on absolute analyte concentration level minimums over certain periods of time. In certain embodiments, analyte trends may be determined based on absolute maximum analyte concentration levels over certain periods of time. In certain embodiments, analyte trends may be determined based on analyte concentration level rates of change over certain periods of time. In certain embodiments, analyte trends may be determined based on analyte baseline rates of change over certain periods of time. In certain embodiments, analyte trends may be determined based on calculated analyte clearance rates over certain periods of time.

With respect to diabetes, CAM system 200 may be configured to measure interstitial glucose levels, generate glucose measurement data, and transmit the sensor data packages to display device 150, and then DSE 114 and DAM 116 may determine various glucose-related data. DSE 114 and DAM 116 may be hosted by network computing device 142 or display device 150.

In certain embodiments, glucose concentration level rates of change may be determined from glucose measurement data. For example, a glucose concentration level rate of change refers to a rate that indicates how time-stamped glucose measurement data values change in relation to one or more other time-stamped glucose measurement data values. Glucose concentration level rates of change may be determined over one or more seconds, minutes, hours, days, etc.

In certain embodiments, a glucose trend may be determined based on glucose measurement data over a certain period of time. In certain embodiments, glucose trends may be determined based on glucose concentration level rates of change over certain periods of time.

In certain embodiments, glycemic variability may be determined from glucose measurement data. For example, glycemic variability refers to a standard deviation of glucose concentration levels over a period of time. Glycemic variability may be determined over one or more minutes, hours, days, etc.

In certain embodiments, a glucose clearance rate may be determined from glucose measurement data following consumption of a known, or estimated, amount of glucose or known nutrient resulting in production of glucose. Glucose clearance rates analyzed over time may be indicative of glucose homeostasis. The glucose clearance rate may be indicative of an effectiveness of a medication type, dosage, and/or frequency.

In certain embodiments, the glucose clearance rate may be determined by calculating a slope between an initial high glucose concentration level (such as a highest glucose concentration level during a period of 20-30 minutes after consumption of glucose) at t₀ and a subsequent low glucose concentration level at t₁. The low glucose concentration level (G_{L}) may be determined based on a user's initial high glucose concentration level (G_{H}) and a baseline glucose concentration level (G_{B}) before consumption of glucose. In certain embodiments, G_{L} can be a glucose concentration level between G_{H} and G_{B}, such as G_{L}=G_{B}+K*(G_{H}−G_{B})/2, where K can be a percentage representing by how much a user's glucose concentration level returned to user's baseline value. When K equals zero, low glucose concentration level equals baseline glucose value. When K equals 0.5, low glucose concentration level equals mean glucose concentration level between initial glucose concentration level and baseline glucose concentration level.

In certain embodiments, the glucose clearance rate may be determined over one or more periods of time after consumption of glucose, such as following an oral glucose tolerance test (OGTT). The glucose clearance rate may be calculated for each time period to represent dynamics of glucose clearance rate after consumption of glucose. These glucose clearance rates calculated over time may be time-stamped and stored in user's profile 118. Certain metrics may be derived from time-stamped glucose clearance rates, such as mean, median, standard deviation, percentile, etc.

In certain embodiments, health and sickness metrics may be determined, for example, based on one or more of user input (such as pregnancy information, known sickness or disease information, etc.), from physiologic sensors (such as temperature, etc.), activity sensors, etc. In certain embodiments, based on values of health and sickness metrics, a user's state may be defined as being one or more of healthy, ill, rested, or exhausted.

In certain embodiments, meal state metric may indicate state user is in with respect to food consumption. For example, meal state may indicate whether user is in one of a fasting state, pre-meal state, cating state, post-meal response state, or stable state. In certain embodiments, meal state may also indicate nourishment on board, such as meals, snacks, or beverages consumed, and may be determined, for example from food consumption information, time of meal information, and/or digestive rate information, which may be correlated to food type, quantity, and/or sequence (such as which food/beverage was caten first).

In certain embodiments, meal habits metrics are based on content and timing of a user's meals. For example, if a meal habit metric is on a scale of 0 to 1, better/healthier meals user cats higher meal habit metric of user will be to 1, in an example. Also, more user's food consumption adheres to a certain time schedule or a recommended diet, closer their meal habit metric will be to 1, in an example.

In certain embodiments, an activity level metric may indicate user's level of activity. In certain embodiments, the activity level metric may be determined based on input from an activity sensor or other physiologic sensors, such as non-analyte sensors 230. In certain embodiments, activity level metric may be calculated by DAM 116 based on input data 128, such as one or more of exercise information, non-analyte sensor data (such as accelerometer data, etc.), time, user input, etc. In certain embodiments, the activity level metric may be expressed as a step rate of user. Activity level metrics may be time-stamped so that they may be correlated with one or more of the user's analyte levels at the same time.

In certain embodiments, body temperature metrics may be calculated by DAM 116 based on input data 128, and more specifically, non-analyte sensor data from a temperature sensor. In certain embodiments, heart rate metrics (such as heart rate and heart rate variability) may be calculated by DAM 116 based on input data 128, such as non-analyte sensor data from a heart rate sensor, etc. In certain embodiments, respiratory metrics (not shown) may be calculated by DAM 116 based on input data 128, such as non-analyte sensor data from a respiratory rate sensor, etc. In certain embodiments, blood pressure metrics (such as blood pressure levels and blood pressure trends) may be calculated by DAM 116 based on input data 128, such as non-analyte sensor data from blood pressure sensor, etc.

In certain embodiments, physiological metrics (such as analyte concentration levels, analyte concentration level rates of change, heart rate, blood pressure, etc.) associated with user may be stored as metric data 130 when a state or condition of user is confirmed. In certain embodiments, such physiological metrics may be analyzed over time to provide an indication of changes in state or condition of user.

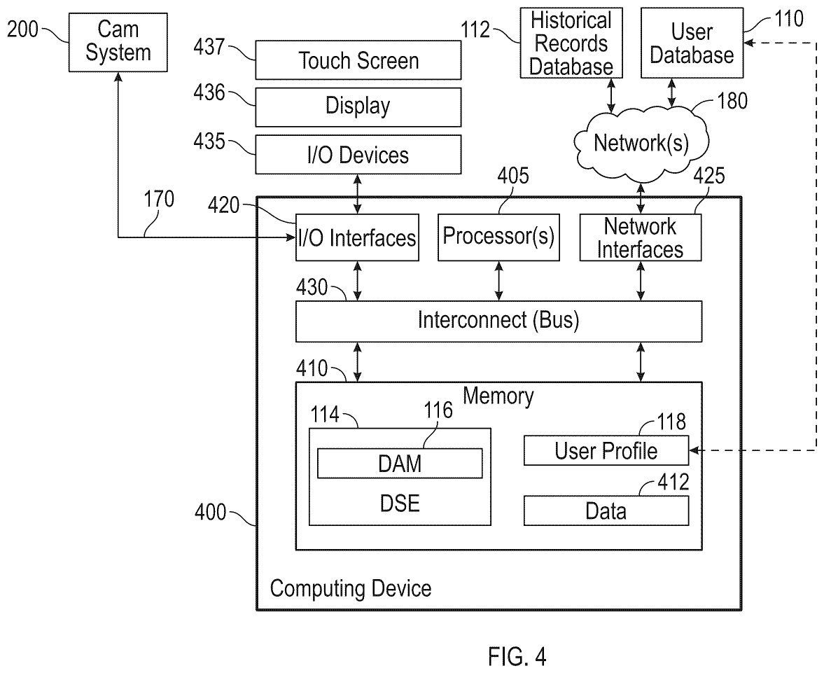

FIG. 4 depicts a block diagram of computing device 400, in accordance with embodiments of the present disclosure.

In certain embodiments, computing device 400 may be configured as display device 150. In these embodiments, computing device 400 may be coupled to network 180 via a wireless connection. Certain display devices 150, such as laptop computers, may include one or more I/O devices 435, such as a keyboard, a mouse, display 436, touch screen 437, etc. Other display devices 150, such as handheld health monitors, smartphones, smartwatches, tablet computers, etc., may include touch screen 437, which is a combination of an I/O device and a display. Other display devices 150, such as wearable health monitors, etc., may include one or more I/O devices 435 (such as buttons, a touchpad, etc.), and display 436 or touch screen 437. Generally, display devices 150 may be battery-powered, and the battery may be periodically recharged or replaced as needed.

In other embodiments, computing device 400 may be configured as network computing device 142, as well as the network computing device(s) of training system 140. In these embodiments, computing device 400 may be coupled to network 180 via a wired or wireless connection, and may include one or more optional I/O devices 435, such as a keyboard, a mouse, display 436, etc.

Computing device 400 includes interconnect (bus) 430 coupled to one or more processors 405, storage element or memory 410, one or more network interfaces 425, and one or more I/O interfaces 420, which may include a display interface (such as HDMI, etc.), a keyboard interface (such as USB, etc.), a local wireless communications interface (such as Bluetooth, BLE, RFID, NFC, etc.), a touch screen interface, etc. In certain embodiments, processor 405 may be a central processing unit (CPU), and computing device 400 may include one or more specialized processors, such as a graphics processing unit (GPU), a neural processing unit (NPU), etc. Generally, network interfaces 425 are coupled to network 180 using a wired or wireless connection(s), and I/O interfaces 420 are coupled to I/O device(s) 435, such as display 436, etc., using wired or wireless connections.

Bus 430 is a communication system that transfers data between processor 405, memory 410, network interfaces 425, and I/O interfaces 420. In certain embodiments, bus 430 transfers data between these components and one or more specialized processors, such as GPUs, NPUs, etc.

Processor 405 includes one or more general-purpose or application-specific microprocessors with one or more processing cores that execute instructions to perform various functions for computing device 400, such as control, computation, input/output, etc. Processor 405 may include a single integrated circuit, such as a micro-processing device, or multiple integrated circuit devices and/or circuit boards working in cooperation to accomplish the appropriate functionality. Additionally, processor 405 may execute software applications and software modules stored within memory 410, such as an operating system, DSE 114, etc. For example, DSE 114 may include rule-based models, machine learning models including LR models, ANNs, recurrent neural networks (RNNs), long short-term memory (LSTM) networks, convolutional neural networks (CNNs), etc., DAM 116, as well as other software modules.

Generally, memory 410 stores instructions for execution by processor 405 as well as data. Memory 410 may include a variety of non-transitory computer-readable medium that may be accessed by processor 405 as well as other components. In various embodiments, memory 410 may include volatile and nonvolatile medium, non-removable medium and/or removable medium. For example, memory 410 may include combinations of random access memory (RAM), dynamic RAM (DRAM), static RAM (SRAM), read only memory (ROM), flash memory, cache memory, and/or any other type of non-transitory computer-readable medium.

Memory 410 contains various components for retrieving, presenting, modifying, and storing user profile 118 as well as other data 412. For example, memory 410 stores software applications and modules that provide functionality when executed by processor 405, such as DSE 114, DAM 116, etc. The operating system provides operating system functionality for computing device 400. Data 412 may include data associated with the operating system, the software applications and modules, DSE 114, DAM 116, etc.

Network interfaces 425 are configured to transmit data to and from network 180 using one or more wired and/or wireless connections. As discussed above, network 180 may include one or more LANs, WLANs, LPWANs, WANs, cellular networks (such as 3G, 4G, LTE, 5G, 6G, etc.), the Internet, etc., employing various network topologies and protocols. For example, network 180 may also include various combinations of wired and/or wireless physical layers, such as, for example, copper wire or coaxial cable networks, fiber optic networks, WiFi networks, Bluetooth mesh networks, CDMA, FDMA and TDMA cellular networks, etc.

I/O interfaces 420 are configured to transmit and/or receive data from I/O devices 435. I/O interfaces 420 enable connectivity between processor 405, memory 410 and I/O device(s) 435 by encoding data to be sent from processor 405 or memory 410 to I/O devices 435, and decoding data received from I/O devices 435 for processor 405 or memory 410. Generally, data may be sent over wired and/or wireless connections. For example, I/O interfaces 420 may include one or more wired communications interfaces, such as USB, Ethernet, etc., and/or one or more wireless communications interfaces, coupled to one or more antennas, such as WiFi, Bluetooth, cellular, etc. Importantly, CAM system 200 may communicate with I/O interfaces 420 via Bluetooth, BLE, RFID, NFC, etc.

Generally, I/O devices 435 provide data to and from computing device 400. As discussed above, I/O devices 435 are operably connected to computing device 400 using a wired and/or wireless connection. I/O devices 435 may include a local processor coupled to a communication interface that is configured to communicate with computing device 400 using the wired and/or wireless connection. For example, I/O devices 435 may include display 436, touch screen 437, a keyboard, a mouse, a touch pad, etc.

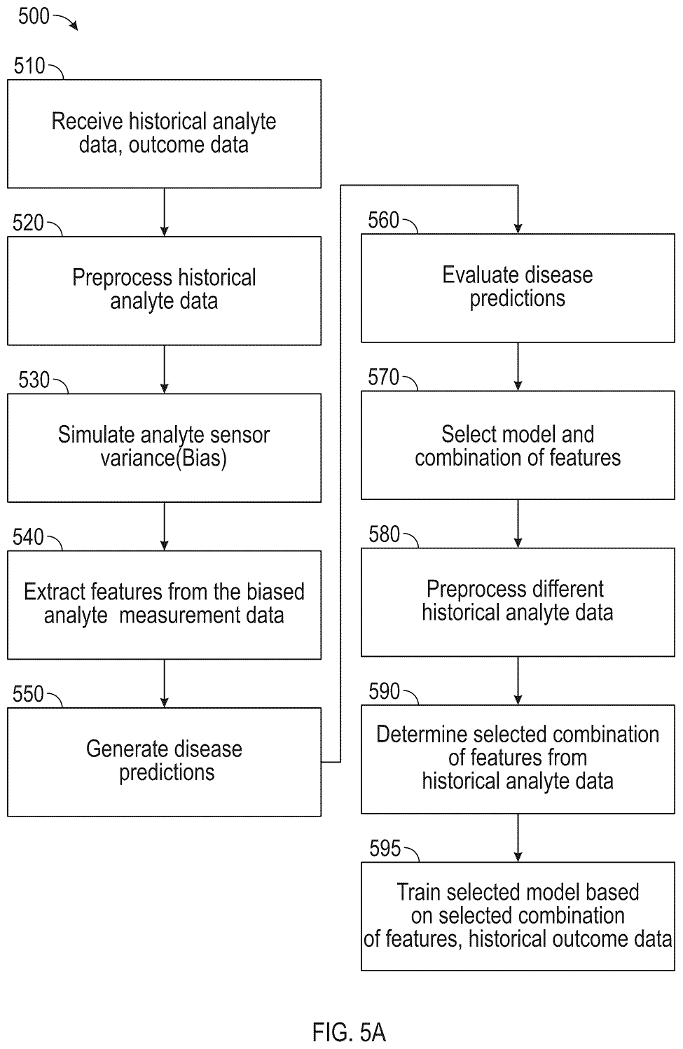

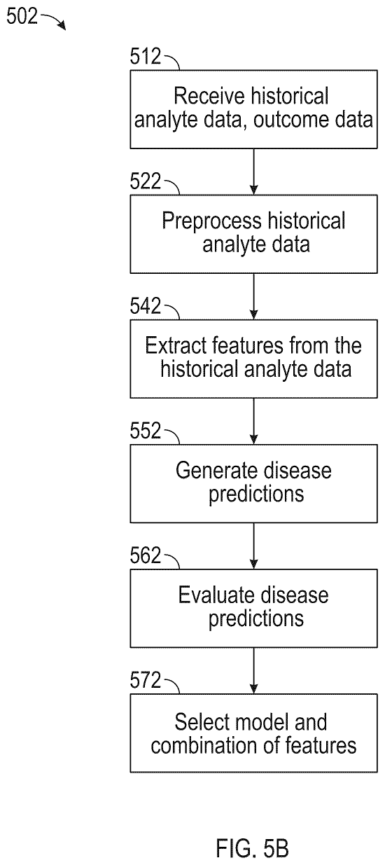

FIG. 5A depicts a process flow diagram 500 for evaluating and selecting a model and a combination of analyte features for predicting disease, in accordance with embodiments of the present disclosure.

Generally, training system 140 is configured to execute, inter alia, the operations represented by process flow diagram 500. These operations may be expressed within one or more software applications and supporting modules that are stored and executed by the network computing device(s) of training system 140. In certain embodiments, the software applications may include a prediction system application (the “prediction system”) and a model manager application (the “model manager”), while the supporting modules may include a preprocessing manager module, a variability simulator module, a feature constructor module, a predictor module, an evaluator module, etc. Other software architectures are also supported. In some embodiments, certain operations may be implemented in hardware, such as ASICs, FPGAs, logic circuitry, etc.

At 510, training system 140 receives historical analyte data and historical outcome data from historical records database 112.

As discussed above, the historical analyte data may include measured analyte concentration levels for a user population, and the historical outcome data may include clinical disease diagnoses that indicate whether each user of the population has been clinically diagnosed with the particular disease based on one or more independent sources.

In certain embodiments, the model manager may be configured to, inter alia, evaluate and select features of the historical analyte data that are robust for accurately predicting a particular disease in presence of variabilities that are caused by manufacturing-related variability of CAS 210, as discussed below. The model manager may include, or be assisted by, the preprocessing manager module, the variability simulator module, the feature constructor module, the predictor module, the evaluator module, as well as other modules or functionality. One or more of these modules may also be incorporated into other process flows, such as process flow diagram 800 for predicting diabetes (as discussed below).

At 520, training system 140 preprocesses the historical analyte data.

In certain embodiments, the preprocessing manager module may be configured to, inter alia, preprocess the historical analyte data to generate a time-ordered sequence of historical analyte data according to respective timestamps. Due to corruption and communication errors, the historical analyte data stored in historical records database 112 may not only be out of time order but may also be missing one or more analyte concentration level measurements. For example, there may be gaps in the time-ordered sequence where one or more analyte concentration level measurements are expected. In these situations, the preprocessing manager module may be further configured to interpolate missing analyte concentration level measurements and incorporate them into time-ordered historical analyte data sequence. The preprocessing manager module may also be configured to filter out portions of the analyte concentration level measurements according to particular criteria, such as to remove corrupted or poor signal quality data. Although this functionality is discussed, the historical analyte data may already be in time order, such that ordering and interpolating analyte concentration level measurements are not needed. Accordingly, the time-ordered sequence of historical analyte data includes analyte concentration level measurements in sequential time series format, i.e., time series analyte measurement data also known as analyte traces.

At 530, training system 140 simulates the analyte sensor variance to generate biased analyte measurement data.

In certain embodiments, the variability simulator module may be configured to, inter alia, introduce manufacturing-related analyte sensor variability (bias) into the time series analyte measurement data to generate biased analyte measurement data. In many embodiments, the variability simulator module may perform multiple variability simulations over a number of simulation rounds, each with a different percent of simulated manufacturing-related analyte sensor variability added to the time series analyte measurement data before the data is passed to the feature constructor module. Each variability simulation generates a different biased analyte measurement data set with a different amount of bias (including a data set without bias).

In certain embodiments, the variability simulator module may apply different analyte sensor performance variabilities and characteristics to the time series analyte measurement data during each simulation round. In certain embodiments, the variability simulator module may simulate analyte sensor bias with a fixed variability (such as standard deviation), which is applied to each analyte trace of the time series analyte measurement data. In one example, fixed variability is 8.

The variability simulator module also associates the biased analyte data with the respective historical outcome data.

At 540, training system 140 extracts analyte features from the biased analyte measurement data.

In certain embodiments, the feature constructor module may be configured to, inter alia, extract one or more features or feature vectors from the biased analyte measurement data for evaluation in connection with predicting a particular disease. Generally, the feature constructor module applies one or more processes or functions to the biased analyte measurement data to extract the analyte features. In certain embodiments, each process or function extracts a different feature from the biased analyte measurement data. As discussed above, the extracted analyte features may include, inter alia, trend-related features, time-related and day-related features, variability and stability features, frequency-related features, value-based feature, etc.

As discussed above, each analyte concentration level measurement within the biased analyte measurement data is associated with a point in time and sequenced with respect to time. In other words, a first measured analyte concentration level obtained at an earlier time is arranged before a second measured analyte concentration level obtained at a later time in time series data. This time series arrangement of the biased analyte measurement data advantageously enables the feature constructor module to extract features related to temporal trends and stability of the biased analyte measurement data, which provides candidates for features that are less sensitive or insensitive to manufacturing-related variability of CAS 210.

The trend-related features may include features that describe patterns or trends in the analyte traces over a particular time interval, such as every 5 minutes, every 10 minutes, every 15 minutes, every 30 minutes, every hour, etc. For example, rate-of-change is a trend-related feature that describes the rate-of-change in analyte concentration levels of a given analyte trace over a particular time interval. Statistics associated with rate-of-change, such as standard deviation, coefficient of variation, skew, kurtosis, etc., may also be provided as trend-related features. Autocorrelation is another trend-related feature that describes the degree of similarity between a given analyte trace and a lagged (time delayed) version of itself over successive time intervals, and may provide a measure of how rapidly analyte concentration levels fluctuate as a result of body response. Additionally, autocorrelation mean (ACM) is another trend-related feature that is the mean of different autocorrelation values taken with lags up to 3 samples from 5-minute analyte trace data.

With respect to diabetes, ACM may be insensitive (i.e., not sensitive) to glucose sensor bias. Glucose traces for persons clinically diagnosed with diabetes typically have higher ACMs, which indicates that the glucose concentration level is not fluctuating rapidly due, at least in part, to a slow pancreatic response time. For example, a person clinically diagnosed with diabetes may have a glucose trace autocorrelation that begins at 1.0 for lag 0 (i.e., 0 time periods apart) and decreases to about 0.7 at lag 10 (i.e., 10 time periods apart). In other words, the glucose trace has a degree of similarity with itself that remains high within first 10 time periods. By contrast, a person not clinically diagnosed with diabetes may have a glucose trace autocorrelation that begins at 1.0 for lag 0 (i.e., 0 time periods apart) and decreases to about 0.1 at lag 10 (i.e., 10 time periods apart). In other words, the glucose trace has a degree of similarity with itself that decreases significantly within first 10 time periods.

The time-related and day-related features may include features that describe the dynamics of the analyte traces during the day and day-to-day, such as mean analyte concentration levels on a particular day, mean analyte concentration levels at a particular time of day, rates-of-change in analyte concentration level on a particular day, rates-of-change in analyte concentration level between particular times of day, etc. Time-related and day-related features may also include statistics-by-day and statistics by time-of-day, differences between various statistical means for different days (such as a mean of daily difference), differences between means of analyte traces for different times of day (such as waking hours and sleeping hours), differences between standard deviations of analyte traces for different times of day, etc.

The variability and stability features may include features that describe the degree of variability and stability of the analyte traces. For example, magnitude peak measures peak width and/or height relative to a set point analyte concentration level. For another example, set point frequency (SPF) is a variability and stability feature that provides a measure of how frequently the analyte concentration level is within a range of an analyte set point value, such as the range with respect to the highest analyte concentration level for a particular analyte trace.

With respect to diabetes, SPF may be less sensitive to glucose sensor bias. Glucose traces for persons clinically diagnosed with diabetes typically have lower set point frequencies. For example, a person clinically diagnosed with diabetes has a SPF spectrum that has a lower peak (such as 50%) that occurs at a larger glucose concentration level (such as 150%), as well as a broader glucose concentration level range (such as x2), than a person not clinically diagnosed with diabetes.

The frequency-related features may include features that describe the dominant frequencies of analyte variability within the analyte traces, which are extracted from the analyte traces after transforming the analyte traces from the time domain to the frequency domain. This transformation enables additional information to be extracted from the analyte traces, such as frequencies into which the time-domain data may be decomposed.

The value-based features may include features that describe various statistical measures of the analyte traces, such as mean, median, standard deviation, skew, kurtosis, coefficient of variation, statistical distributions, etc., interquartile range differences, time-based threshold measures, etc. For example, the value-based features may include a time-within-range measure, which corresponds to an amount of time that each analyte trace is between a first analyte concentration level and a second analyte concentration level that is less than first analyte concentration level, corresponding to the upper and lower limits of a range, respectively. As another example, the value-based features may include a time outside range measure, which corresponds to an amount of time that an analyte trace is outside such a range. As another example, the value-based features may include event occurrence-based features, which may indicate occurrences of each analyte trace increasing above the first analyte concentration level (such as a hyperglycemia event) and/or decreasing below the second analyte concentration level (such as a hypoglycemia event). Additional examples of value-based features include analyte concentration levels corresponding to a threshold percentile of the analyte (such as a statistically significant threshold percentile such as 94th percentile or greater), a 10 to 90 percentile analyte range, amplitude-based features (such as mean amplitude of analyte concentration level excursions), etc.

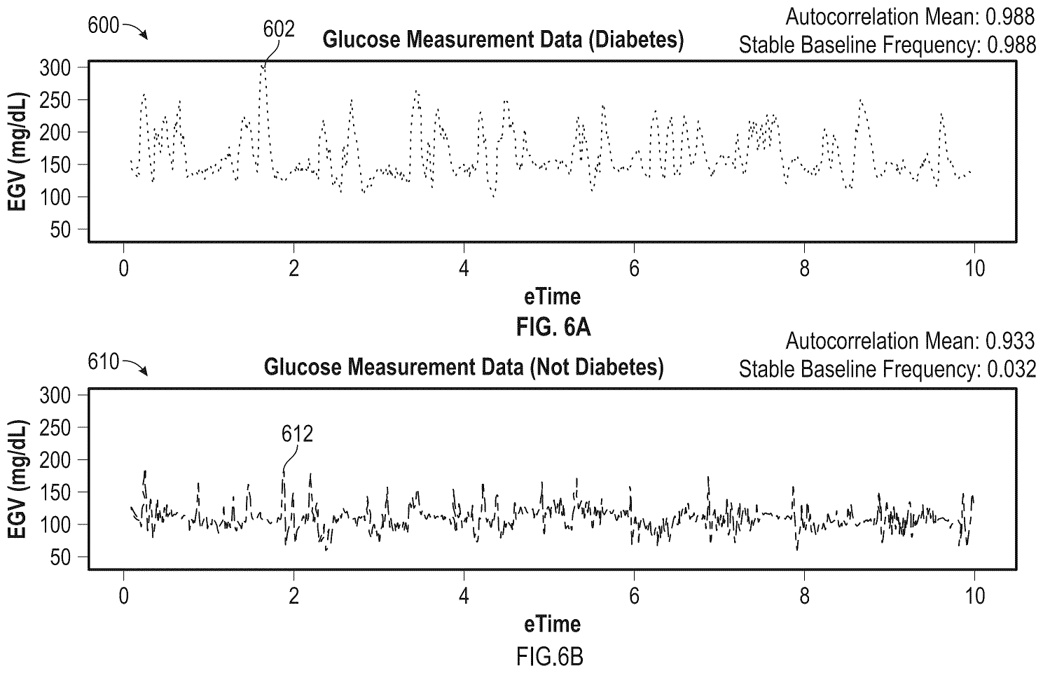

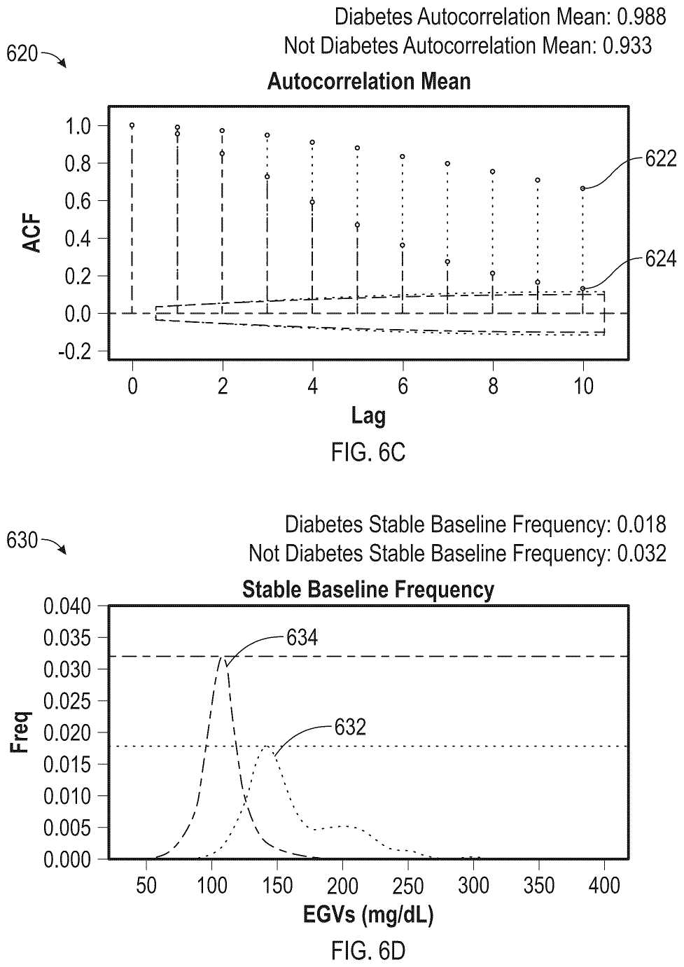

FIGS. 6A and 6B presents glucose measurement data graphs 600 and 610, respectively, in accordance with embodiments of the present disclosure. Glucose trace 602 for a person clinically diagnosed with diabetes is presented as estimated glucose value (EGV) in mg/dL vs. elapsed time (eTime) in hours. Similarly, glucose trace 612 for a person not clinically diagnosed with diabetes is presented as estimated glucose value (EGV) in mg/dL vs. elapsed time (cTime) in hours.

FIG. 6C presents autocorrelation mean graph 620, in accordance with embodiments of the present disclosure. Autocorrelation mean 622 for a person clinically diagnosed with diabetes and autocorrelation mean 624 for a person not clinically diagnosed with diabetes are presented as autocorrelation function (ACF) vs. lag. The diabetes autocorrelation mean feature value is 0.988 and the non-diabetes autocorrelation mean feature value is 0.933.

FIG. 6D presents set point frequency graph 630, in accordance with embodiments of the present disclosure. Set point frequency 632 for a person clinically diagnosed with diabetes and set point frequency 634 for a person not clinically diagnosed with diabetes are presented as frequency (Hz) vs. estimated glucose value (EGV) in mg/dL. The diabetes set point frequency feature value is 0.018 and non-diabetes set point frequency feature value is 0.032.

Referring back to FIG. 5A, at 550, training system 140 generates disease predictions using one or more models and different combinations of the extracted analyte features.

In certain embodiments, the predictor module may be configured to, inter alia, generate disease predictions for a particular disease using one or more models based on different combinations of extracted analyte features from the biased analyte measurement data sets. The disease predictions may include binary disease screening predictions (such as normal or predisposed), as well as binary disease diagnosis predictions (such as normal/predisposed or disease). The predictor module may use bivariate models that combine two features of the extracted analyte features, as well as multivariate models that combine three (or more) features of extracted analyte features. Each disease prediction may be associated with the historical outcome data for a member of the user population, which includes clinical diagnoses such as normal, predisposed, disease, etc. The models may include rule-based models, machine learning (ML) models, etc.

In certain embodiments, a rule-based model may include a simple rule set for a combination of two features may include a first conditional test for the first feature (such as If X>X_{threshold}, Then A=True; If X<X_{threshold}, Then A=True; etc.), a second conditional test for the second feature (such as If Y>Y_{threshold}, Then B=True; If Y<Y_{threshold}, Then B=True; etc.), and a third conditional test for the disease prediction that is based on the outcomes of the first and second conditional tests (such as If A=B=True, Then Disease=True; If A OR B=True, Then Disease=True; etc.). The selection of the combination of features, as well as the parameters, thresholds, etc. within the conditional tests, may be determined using biased analyte data and historical outcome data of the user population.

In one example, the first feature is the ACM feature with a threshold of 0.950, and the second feature is the SPF feature with a threshold of 0.250. The first conditional test may be expressed as: If (X_{ABF}>0.950) Then A=TRUE, the second conditional test may be expressed as: If (Y_{SPF}<0.250) Then B=TRUE, and the third conditional test may be expressed as: If A=B=True, Then Disease=True.

In certain embodiments, an ML model (such as an ANN) may be used to predict disease. An ANN models relationships between input data or signals and output data or signals using a network of interconnected nodes that is trained through a learning process. The nodes are arranged into various layers, including, for example, an input layer, one or more hidden layers, and an output layer. The input layer receives input data, such as, for example, image data, sensor time series data, etc., and output layer generates output data, such as, for example, a probability that image data contains a known object, a medical condition, etc. Each hidden layer provides at least a partial transformation of input data to output data. A deep ANN (DNN) has multiple hidden layers in order to model complex, nonlinear relationships between input data and output data.

In a fully-connected, feedforward ANN, each node is connected to all of nodes in preceding layer, as well as to all of nodes in subsequent layer. For example, each input layer node is connected to each hidden layer node, each hidden layer node is connected to cach input layer node and each output layer node, and each output layer node is connected to each hidden layer node. Additional hidden layers are similarly interconnected. Each connection has a weight value, and each node has an activation function, such as, for example, a linear function, a step function, a sigmoid function, a hyperbolic or tanh operation, a rectified linear unit (ReLu) function, etc., that determines output of node based on weighted sum of inputs to node. The input data propagates from input layer nodes, through respective connection weights to hidden layer nodes, and then through respective connection weights to output layer nodes. The sigmoid and ReLu functions output a number between 0 and 1, while tanh operation outputs a number between −1 and 1, for any given input.

More particularly, at each input node, input data is provided to activation function for that node, and output of activation function is then provided as an input data value to each hidden layer node. At each hidden layer node, input data value received from each input layer node is multiplied by a respective connection weight, and resulting products are summed or accumulated into an activation signal value that is provided to activation function for that node. The output of activation function is then provided as an input data value to each output layer node. At each output layer node, output data value received from each hidden layer node is multiplied by a respective connection weight, and resulting products are summed or accumulated into an activation signal value that is provided to activation function for that node. The output of activation function is then provided as output data. Additional hidden layers may be similarly configured to process data.

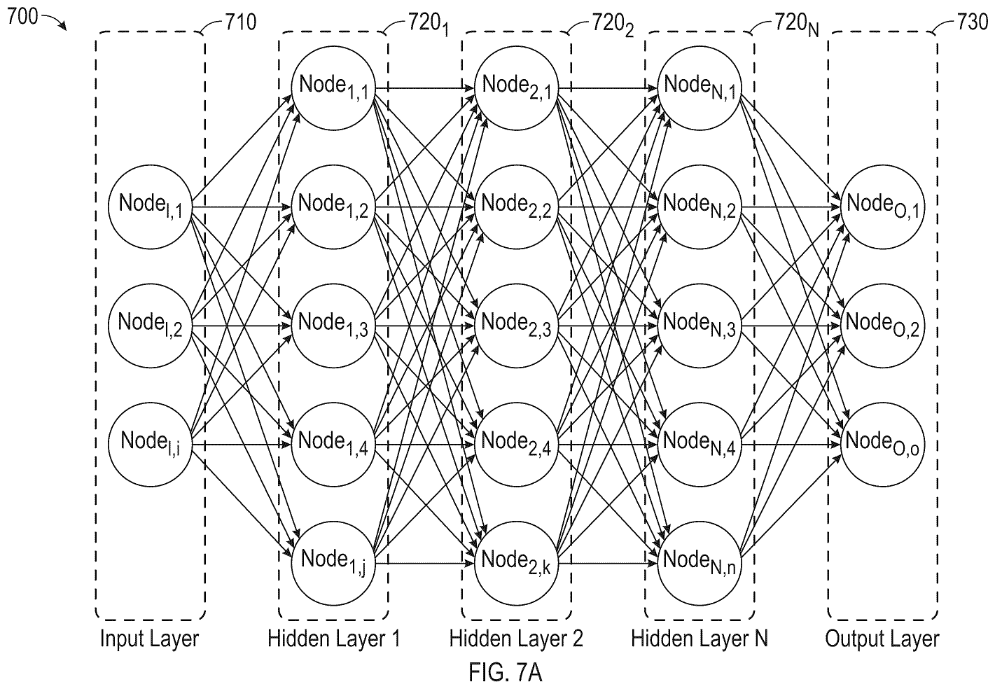

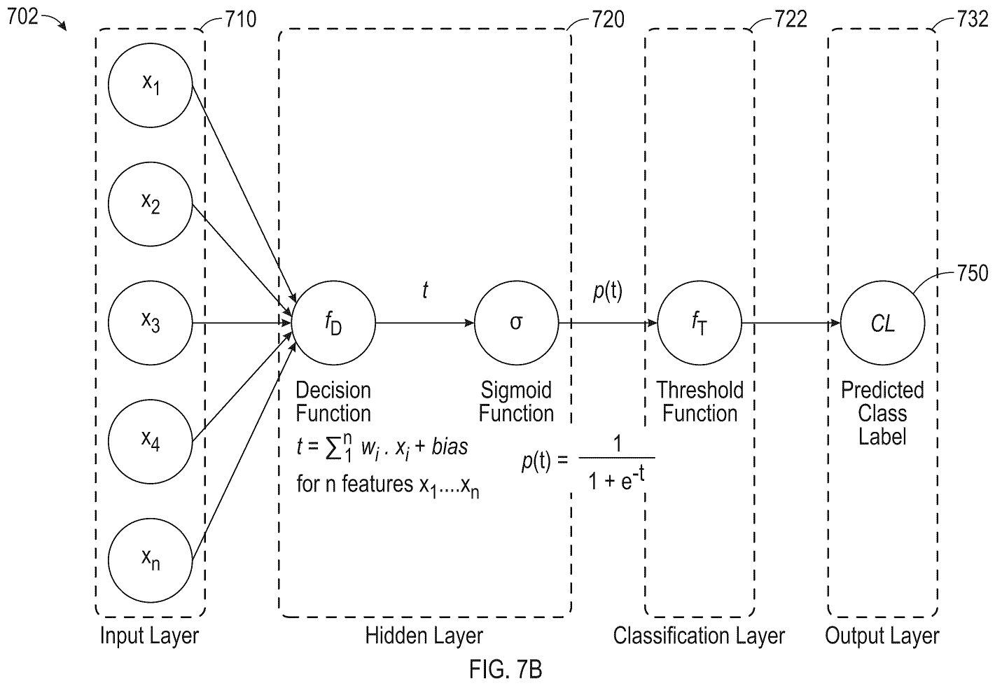

FIG. 7A depicts ANN 700, in accordance with embodiments of the present disclosure.

ANN 700 includes input layer 710, one or more hidden layers, such as hidden layers 710₁, 720₂, . . . , 720_{N}, and output layer 730. Input layer 710 includes one or more input nodes, such as Node_{I,1}, Node_{I,2}, . . . , Node_{I,i}. Hidden layer 720₁ includes one or more hidden nodes, such as Node_{1,1}, Node_{1,2}, . . . , Node_{1,j}. Hidden layer 720₂ includes one or more hidden nodes, such as Node_{2,1}, Node_{2,2}, . . . , Node_{2,k}. Hidden layer 720_{N} includes one or more hidden nodes, such as Node_{N,1}, Node_{N,2}, . . . , Node_{N,n}. Output layer 730 includes one or more output nodes, such as Node_{O,1}, Node_{O,2}, . . . , Node_{O,o}. In example depicted in FIG. 7A, there are N hidden layers; input layer 710 includes “i” nodes, hidden layer 730₁ includes “j” nodes, hidden layer 720₂ includes “k” nodes, hidden layer 730_{N} includes “n” nodes, and output layer 730 includes “o” nodes.

In certain embodiments, N equals 3, “i” equals 3, “j”, “k” and “n” equal 5 and “o” equals 3. Input Node_{I,1}, Node_{I,2} and Node_{I,3} are each coupled to hidden Node_{1,1}, Node_{1,2}, Node_{1,3}, Node_{1,4} and Node_{1,5}. Hidden Node_{1,1}, Node_{1,2}, Node_{1,3}, Node_{1,4} and Node_{1,5} are cach coupled to hidden Node_{2,1}, Node_{2,2}, Node_{2,3}, Node_{2,4} and Node_{2,5}. Hidden Node_{2,1}, Node_{2,2}, Node_{2,3}, Node_{2,4} and Node_{2,5} are each coupled to hidden Node_{3,1}, Node_{3,2}, Node_{3,3}, Node_{3,4} and Node_{3,5}. Hidden Node_{3,1}, Node_{3,2}, Node_{3,3}, Node_{3,4} and Node_{3,5} are each coupled to output Node_{O,1}, Node_{O,2}, Node_{O,3}.

Many other variations of input, hidden and output layers are clearly possible, including hidden layers that are locally-connected, rather than fully-connected, to one another.

Training an ANN includes optimizing connection weights between nodes by minimizing prediction error of output data until ANN achieves a particular level of accuracy. One method is backpropagation, or backward propagation of errors, which iteratively and recursively determines a gradient (i.e., a partial derivative of error function) with respect to each weight, and then adjusts each weight to improve performance of network.

FIG. 7B depicts LR model 702, in accordance with embodiments of the present disclosure.

Generally, LR model 702 may be described as including input layer 710, hidden layer 720, classification layer 722 and output layer 730. Input layer 710 receives a set of input features x₁ . . . , xₙ. LR model 702 is a univariate LR model when set of input features includes a single input feature, a bivariate LR model when set of input features includes two input features, and a multivariate LR model when set of input features includes three or more input features. Hidden layer 720 includes a decision function (f_{D}) and a sigmoid function (σ), classification layer 722 includes a threshold function (f_{T}), and output layer 730 generates and outputs predicted class label 750, such as “disease” or “no disease.” Predicted class label 750 is the disease prediction.

Hidden layer 720 calculates the probability of disease p(t) for a set of input features (xₙ) based on a sigmoid function (σ) that operates on the output (t) of the decision function (f_{D}). Classification layer 722 applies a threshold function (f_{T}) to the probability of disease p(t). The threshold function (f_{T}) may include a probability threshold against which the probability of disease p(t) is compared. In certain embodiments, the threshold function (f_{T}) may output a value of 0 when the probability of disease p(t) is less than the probability threshold, and output a value of 1 when the probability of disease p(t) is equal to or greater than the probability threshold. Output layer 732 generates the predicted class label, i.e., “disease” or “no disease,” based on the output of the threshold function (f_{T}).

The decision function (f_{D}) generates output (t) based on set of input features (xₙ), weights (wₙ), and a bias, and is given by Equation 1:

$$t = {\sum }_{1}^{n} {w}_{i} \cdot {x}_{i} + bias \qquad Eq . 1$$

The sigmoid function (σ) generates the probability of the disease p(t) and is given by Equation 2:

$$p ( t ) = \frac{1}{1 + {e}^{- t}} \qquad Eq . 2$$

Generally, the threshold function (f_{T}) may be used to diagnose whether the user has the disease or does not have the disease. In certain embodiments, the threshold function (f_{T}) may also be used to screen whether the user has a predisposition for the disease or does not have a predisposition for the disease.

For example, a diabetes screening model may include a screening threshold function (f_{TS}) that determines whether the user has a predisposition for diabetes (“pre-diabetes”), while a diabetes diagnosis model may include a diagnostic threshold function (f_{TD}) that determines whether the user has diabetes. The probability threshold for the screening threshold function (f_{TS}) is less than the probability threshold for the diagnostic threshold function (f_{TD}).

Referring back to FIG. 5A, at 560, training system 140 evaluates the disease predictions.

In certain embodiments, the evaluator module may be configured to, inter alia, categorize each different combination of features extracted from the biased analyte measurement data based on a performance metric and a robustness metric. The performance metric may indicate the classification or prediction accuracy of a feature based on the disease predictions and the historical outcome data. The robustness metric may indicate the insensitivity of a feature to the simulated manufacturing-related analyte sensor variability.

The performance metric may include, inter alia, a true positive rate (TPR) and a true negative rate (TNR). The true positive rate provides the percentage of disease predictions that correctly predict a disease condition (i.e., sensitivity or probability of detection). The true negative rate provides the percentage of disease predictions that correctly predict a non-disease condition (i.e., specificity). The performance metric may also include a false positive rate (FPR) that provides the percentage of disease predictions that incorrectly predict a disease condition (i.e., probability of false alarm), and a false negative rate (FNR) that provides the percentage of disease predictions that incorrectly predict a non-disease condition (i.e., miss rate). The performance metric may also include a positive predictive value (PPV) that provides the percentage of positive results that are truly positive (PPV=TP/(TP+FP)), and a negative predictive value (NPV) that provides the percentage of negative results that are truly negative (NPV=TN/(TN+FN)).

The performance metric may also include a receiver operating characteristic (ROC) curve and an area under curve (AUC). The ROC curve plots the true positive rate (TPR) against the false positive rate (FPR), described above, at various threshold settings. The AUC is the area under the ROC curve, and is equal to the probability that a classifier will rank a randomly chosen positive prediction higher than a randomly chosen negative prediction. In other words, AUC is the probability that the classifier will be able to distinguish between a randomly selected positive prediction and a randomly selected negative prediction.

The higher the performance metric, the higher that sensitivity and specificity for predicting the disease. For example, the performance metric may rank features on a pre-defined scale, such as a scale from 0 to 1, where 0 refers to none (such as 0%) of the corresponding model predictions being accurate and 1 refers to all (such as 100%) of the corresponding model predictions being accurate.

The robustness metric may indicate the degree to which the performance metric changes due to the amount of bias (such as the % variability) that is added to the time series analyte measurement data (which may be averaged across repetitions for each member of the user population). For example, the robustness metric may combine bias sensitivity for true positive rate and true negative rate. The robustness metric may rank the features on a pre-defined scale having a highest value and a lowest value. The highest value may indicate no change in the performance metric in response to the simulated bias, while the lowest value may indicate a maximum change in the performance metric in response to the simulated variability. The higher the robustness metric, the more insensitive the feature may be to manufacturing variations of CAS 210. In some examples, in addition to, or as an alternative to, the robustness metric, the evaluator module may determine a variability sensitivity metric, which may be an inverse of the robustness metric. For example, the higher the variability sensitivity metric, the more sensitive (and less robust) the feature may be to manufacturing variations in CAS 210. In general, highly robust analyte features (such as those having a high robustness metric or a low variability sensitivity metric) correspond to extracted analyte features that exhibit little change in the performance metric with various amounts of simulated variability (bias). For example, trend-related features and variability and stability features may produce extracted analyte features that have relatively high robustness metrics (and relatively low variability sensitivity metrics).

At 570, training system 140 selects the model and the combination of features.

The evaluator module may be further configured to, inter alia, select a model and a combination of analyte features based on the robustness metric and the performance metric. The selected feature combination balances performance and robustness for a model that accurately predicts the disease with high sensitivity and specificity but is relatively unaffected by manufacturing-related variability that affects output and performance of CAS 210. Importantly, a model built from a combination of two or more features provides a combination of robustness and performance that is greater than that provided by either feature alone. For example, when a first feature has a higher performance metric than a second feature, and a second feature has a higher robustness metric than first feature, the combination of the first and second features provides a higher overall performance than either feature alone.

Certain value-based features may have relatively high performance metrics for certain diseases, while other value-based features may be relatively sensitive to manufacturing-related variability of CAS 210. For example, increasing simulated positive sensor bias, which raises the concentration levels of the analyte traces, may decrease true negative rate (and increase false positive rate) because the models may incorrectly predict that certain members of the user population without the disease actually have the disease due to the elevated concentration levels of the associated analyte traces. Similarly, increasing simulated negative sensor bias, which lowers the concentration levels of the analyte traces, may decrease true positive rate (and increase false negative rate) because the models may incorrectly predict that certain members of the user population with the disease actually do not have the disease due to reduced concentration levels of the associated analyte traces. Accordingly, the value-based features may be combined with different features that have a high robustness metric, such as the trend-related features, in the robust analyte feature combination 434.

Generally, the selected combination of analyte features may include any type of feature. For example, all of the features may be selected from the trend-related features, one feature may be selected from the trend-related features, such as autocorrelation mean, and one feature may be selected from the variability and stability features, such as set point frequency, etc.

In certain embodiments, the evaluator module may filter the disease predictions to identify the model and feature combinations with robustness metrics that are greater than a robust threshold value, and then select the model and feature combination with the highest performance metric from the filtered model predictions. In other embodiments, the evaluator module may filter the disease predictions to identify the model and feature combination with performance metrics that are greater than a performance threshold value, and then select the model and feature combination with the highest robustness metric from the filtered model predictions.

At 580, training system 140 preprocesses different historical analyte data.

As described above, the preprocessing manager module may be configured to, inter alia, preprocess different historical analyte data to generate a different time-ordered sequence of historical analyte data. In other words, the time-ordered sequence of historical analyte data generated at 580 is different than the time-ordered sequence of historical analyte data generated at 520.

At 590, training system 140 determines the selected combination of features from the time-ordered sequence of different historical analyte data.

In certain embodiments, the feature constructor module may be configured to, inter alia, determine the selected combination of features from the historical analyte measurement data. As described above, the feature constructor module applies the relevant processes or functions to the historical analyte measurement data to determine the combination of analyte features.

At 595, training system 140 trains the selected model based on the selected combination of features from the time-ordered sequence of different historical analyte data, and the historical outcome data.

In certain embodiments, the selected model may be an LR model, an ANN, etc., that is trained using supervised learning. In other embodiments, the selected model may include an ensemble of models, such as an ensemble of LR models, each one trained to predict diabetes based on a particular feature the selected combination of features. The ensemble may include the same type of machine learning model, and each machine learning model may be trained using the same technique. Alternatively, the ensemble may include different types of machine learning models, and each type of machine learning model may be trained using the same technique or a different technique, such as supervised learning, unsupervised learning, reinforcement learning, etc.

In certain embodiments, the model manager may build and train a bivariate LR model which includes a combination of two features, such as the ACM feature and the SPF feature. Given the selected combination of features and the clinical disease diagnoses from the historical outcome data, the model manager may use one or more approaches for “fitting” these data to an equation for the bivariate LR model to produce the disease prediction within some tolerance. In many embodiments, the logarithmic (“log”) loss cost function may be minimized to fit these data to an equation for bivariate LR model. Other examples of such fitting approaches may include a least squares approach, a least absolute deviations regression, minimizing a penalized version of least squares cost function (such as ridge regression or lasso), etc. By “fitting” it is meant that the model manager estimates the bivariate LR model parameters for the equation using one or more approaches and these data.

In one embodiment, the first feature (i.e., independent variable) may be the ACM feature (x_{ACM}), the second feature (i.e., independent variable) may be the SPF feature (x_{SPF}), and the decision function (f_{D}) for sigmoid function (Equation 2) is given by Equation 3:

$$t = bias + {w}_{ACM} \cdot {x}_{ACM} + {w}_{SPF} \cdot {x}_{SPF} \qquad Eq . 3$$

The bivariate LR model parameters include the weights (i.e., w_{ACM} and WSPF in Eq. 3) and a bias (i.e., bias in Eq. 3). In one example, historical glucose measurements and related clinical diabetes diagnoses yielded the following bivariate LR model parameters: w_{ACM} equal to 2.9, w_{SPF} equal to −2.4 and a bias equal to −4.5. In certain embodiments, the prediction system may input the feature combination into the bivariate LR model as one or more vectors or a matrix, the bivariate LR model may then apply the weights and the bias to these input values, and then output the disease prediction as output data 144.

In certain embodiments, the model manager may build and train a multivariate LR model that includes a combination of three or more selected features. In other embodiments, the model manager may build and train an ANN, etc.

Generally, training the model includes providing a portion of the selected combination of historical features and the related historical outcome data to the model as a training data instance, receiving disease predictions from the model, comparing the disease predictions to the historical outcome data (such as the clinical disease diagnoses) using a loss function (such as mean squared error, etc.), adjusting the weights of the model based on the comparison, and then repeating the process for many training iterations. Each training iteration processes a different portion of the selected combination of historical features and the related historical outcome data, i.e., a different training data instance.

The model manager may perform these iterations until the model generates disease predictions that consistently and substantially match the historical outcome data. The capability of a machine learning model, such as an LR model or an ANN, to consistently generate predictions that substantially match expected output portions may be referred to as “convergence.” In other words, model manager trains machine learning model until model “converges” on a solution in which weights of model have been sufficiently adjusted during training iterations so that final values of weights consistently generate predictions that substantially match expected output portions.

In certain embodiments, the model may be configured to receive and process other data in addition to the selected combination of historical features and the related historical outcome data during training. For example, the model manager may add additional data to the training data instances that describes other aspects of the user population, such as demographic information, medical history, exercise, stress, etc. In response, the model may generate a disease prediction in a similar manner as discussed above, such that the disease prediction may be compared to the historical outcome data (such as the clinical disease diagnoses), and the weights of the model adjusted based on the comparison, etc.

In certain other embodiments, the historical analyte data may include inherent analyte sensor bias that sufficiently reflects manufacturing-related variability. Accordingly, the model may be developed directly from the historical analyte data without the introduction of additional analyte sensor bias.

FIG. 5B depicts a process flow diagram 502 for evaluating and selecting a model and a combination of analyte features for predicting disease, in accordance with embodiments of the present disclosure.

At 512, training system 140 receives historical analyte data and historical outcome data from historical records database 112, as described above with respect to 510. The historical analyte data includes inherent analyte sensor bias that sufficiently reflects manufacturing-related variability.

At 522, training system 140 preprocesses the historical analyte data, as described above with respect to 520.

At 542, training system 140 extracts analyte features from the historical analyte data, using the same process described above with respect to 540 and the biased analyte measurement data.

At 552, training system 140 generates disease predictions using one or more models and different combinations of the extracted analyte features, as described above with respect to 550.

At 562, training system 140 evaluates the disease predictions, as described above with respect to 560.

At 572, training system 140 selects the model and the combination of features, as described above with respect to 570.

Advantageously, the model does not need to be further trained using the selected combination of features extracted from additional historical data.

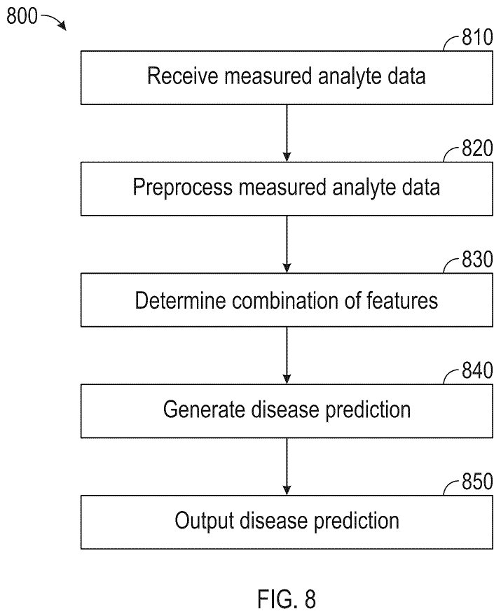

FIG. 8 depicts process flow diagram 800 representing operations for predicting disease, in accordance with embodiments of the present disclosure.

In certain embodiments, network computing device 142 may store and execute the relevant portions of the prediction system to provide a network-based, client-server disease prediction solution. In other embodiments, one of display devices 150, such as smartphone 154, may store and execute the relevant portions of the prediction system to provide a local disease prediction solution rather than a network-based, client-server approach. In some embodiments, in addition to generating and transmitting sensor data packages to a display device 150, CAM system 200 may store and execute the relevant portions of the prediction system to provide another local disease prediction solution.

At 810, the prediction system receives measured analyte data for a user. In certain embodiments, the measured analyte data may be received in sensor data packages transmitted by CAM system 200 worn by the user (or forwarded by a display device 150). In other embodiments, the measured analyte data may be received, in aggregated form, from a display device 150 or from user database 110 for the user.

At 820, the preprocessing manager module may preprocess the measured analyte data to generate a time-ordered sequence of measured analyte data according to respective timestamps, similar to process described above with respect to the historical analyte data (at blocks 520 and 580 of flow process diagram 500).

At 830, the feature constructor module may determine the combination of features from the measured analyte data. More particularly, the feature constructor module may apply the relevant processes or functions to the measured analyte data to determine the combination of analyte features, similar to process described above with respect to the historical analyte data (at block 590 of flow process diagram 500).

In certain embodiments, the prediction system may receive additional data, from a display device 150 or user database 110, that describe different aspects of the user. The additional data may include environmental data (such as temperature, etc.), already-observed adverse effects data (such as data describing that any of a variety of adverse effects associated with the disease that have already been observed, etc.), demographic data (such as age, gender, ethnicity, etc.), medical history data, stress data, nutrition data, exercise data, prescription data, height and weight data, occupation data, etc.

At 840, the trained model generates the disease prediction based on the combination of features determined from the measured analyte data at block 830. As discussed above, the model may be a rule-base model, an ML such as an LR model, etc.

At 860, the prediction system outputs the disease prediction (as output data 144) to a display device 150 associated with the user, to the user database 110, etc.

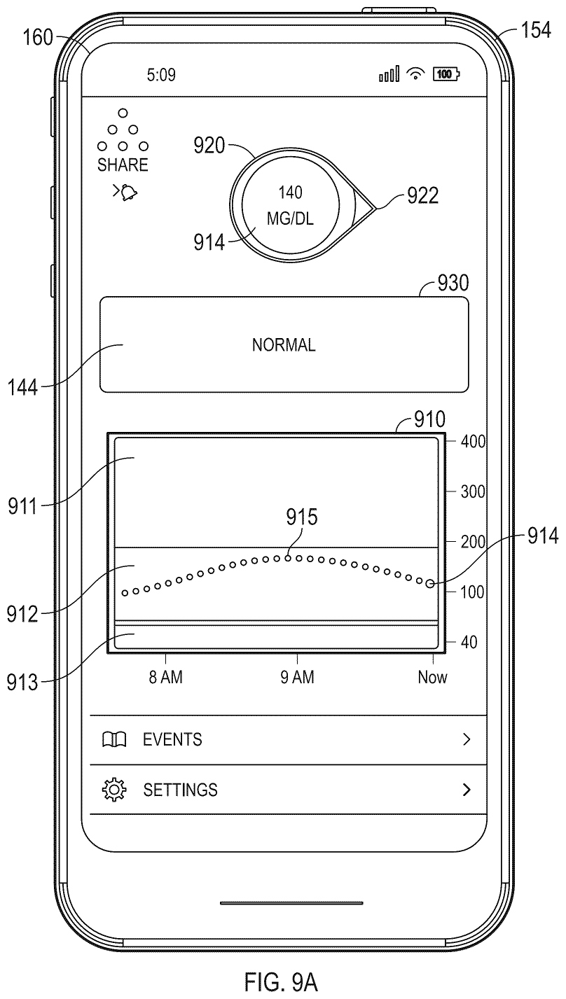

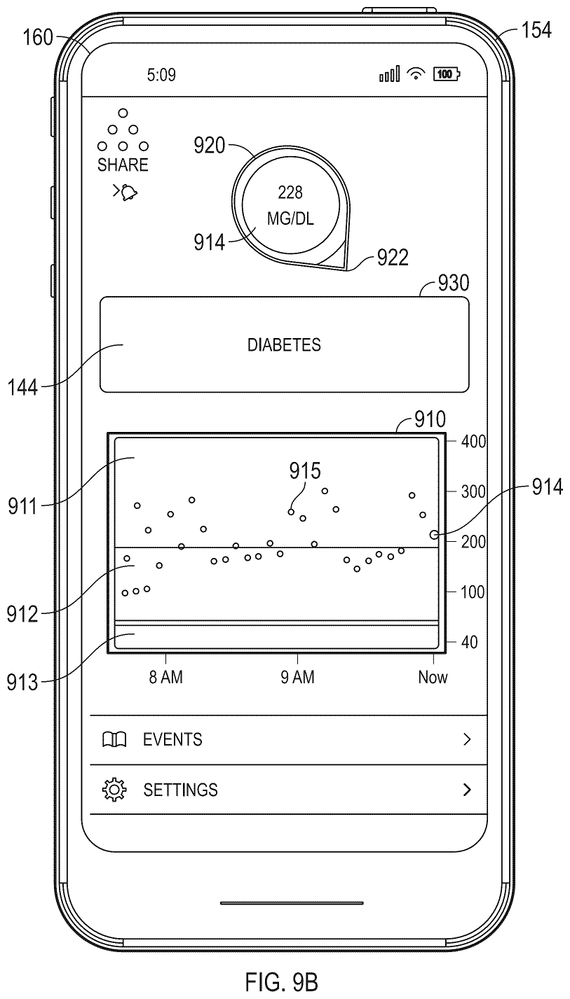

FIGS. 9A and 9B depict GUI 160 for displaying measured analyte data and a disease prediction on smartphone 154, in accordance with embodiments of the present disclosure.

In certain embodiments, GUI 160 includes, inter alia, analyte measurement data graph 910, analyte measurement display widget 920, and disease prediction display widget 930. For convenience, example analyte measurement data values related to glucose concentration levels and example disease predictions related to diabetes are illustrated.

Analyte measurement data graph 910 displays analyte measurement data 915 acquired over a period of time. Most recent analyte measurement 914 may be displayed as a hollow circle or white dot, while the remaining analyte measurements 915 may be displayed as solid circles or black dots; other representations may also be used. In the examples depicted in FIGS. 9A and 9B, glucose concentration levels in units of mg/dL were acquired every 5 minutes and the time period displayed is 2.5 hours; other measurement intervals and time periods may also be used, such as 5 minutes and 3 hours, 10 minutes and 6 hours, etc.

Analyte measurement data graph 910 may also display user-customizable regions including above target range region 911, target range region 912 and below target range region 913. The regions may be defined by one or more user-customizable threshold values. For example, above target range region 911 may be defined by a high threshold value (such as 220 mg/dL), below target range region 913 may be defined by a low threshold value (such as 80 mg/dL), while target range regions 912 may be defined as region between high and low threshold values. Additional thresholds and regions may also be used. In certain embodiments, each region may be color-coded with a different color, such as yellow for above target range region 911, grey for target range region 912, and red for below target range region 913.

Analyte measurement display widget 920 displays the value of most recent analyte measurement 914 as well as trend arrow 922. In certain embodiments, the central portion of analyte measurement display widget 920 may be color-coded to match the region in which most recent analyte measurement 914 is disposed, such as yellow for above target range region 911, grey for target range region 912, and red for below target range region 913. Trend arrow 922 indicates certain trends in a recent number of recent analyte measurement data 915, including a trend direction (i.e., increasing, decreasing or steady analyte measurement levels) and a trend speed (i.e., rate-of-change of analyte measurement levels). For example, trend arrow 922 indicates a steady trend when disposed in a horizontal orientation, trend arrow 922 indicates a rising or falling trend when disposed in a vertical orientation (i.e., up or down, respectively), and trend arrow 922 indicates a slowly rising or falling trend when disposed between horizontal and vertical orientations.

Disease prediction display widget 930 displays output data 144, which may include, inter alia, the disease prediction.

FIG. 9A depicts most recent analyte measurement 914 as disposed within target range region 912 with a value of 140 mg/dL, a slowly decreasing trend arrow 922, and a disease prediction of “normal.” FIG. 9B depicts most recent analyte measurement 914 as disposed within target range region 911 with a value of 228 mg/dL, a falling trend arrow 922, and a disease prediction of “diabetes.”

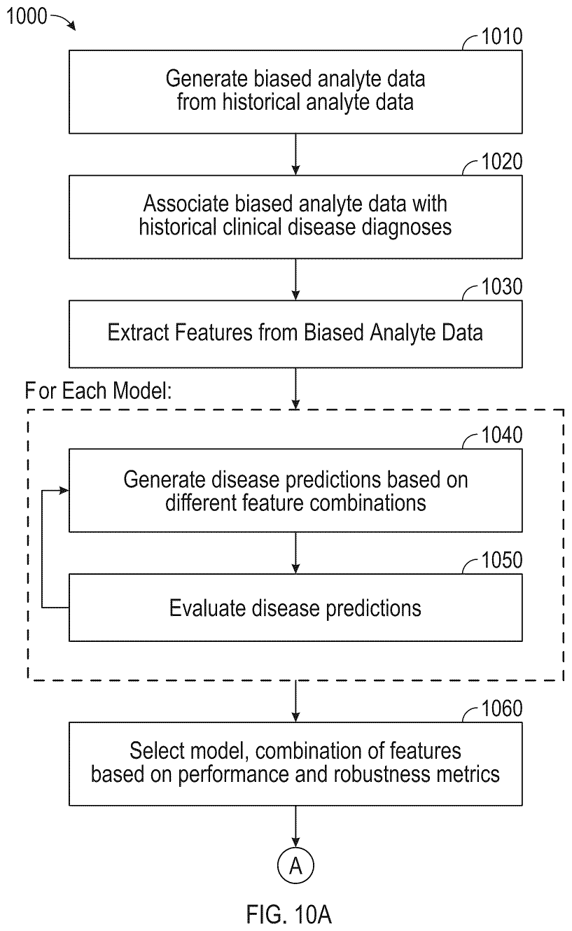

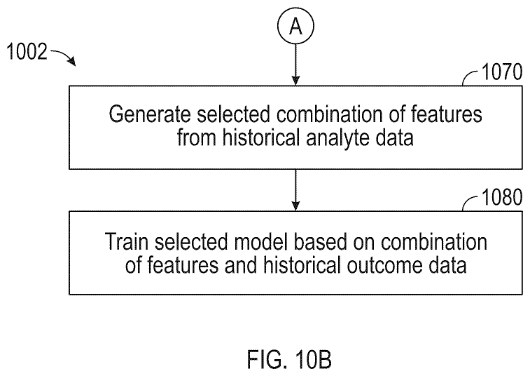

FIG. 10A depicts process flow diagram 1000 representing operations for evaluating and selecting a model and a combination of analyte features for predicting disease, in accordance with embodiments of the present disclosure.

Generally, blocks 1010, 1020, 1030, 1040, 1050, 1060, 1070, and 1080 may be performed by training system 140, in accordance with the processes described above.

At block 1010, biased analyte data are generated by adding analyte sensor bias to historical analyte data.

At block 1020, the biased analyte data are associated with clinical disease diagnoses associated with the historical analyte data.

At block 1030, features are extracted from the biased analyte data. Blocks 1040 and 1050 are repeated for each model under consideration.

At block 1040, disease predictions are generated based on different combinations of the features extracted from the biased analyte data.

At block 1050, the disease predictions are evaluated based on the clinical disease diagnoses associated with the biased analyte data.

At block 1060, a model and a combination of features are selected based on a performance metric and a robustness metric. In certain embodiments, flow continues to FIG. 10B.

FIG. 10B depicts process flow diagram 1000 representing operations for training a model based on a combination of analyte features to predict disease, in accordance with embodiments of the present disclosure.

At 1070, the selected combination of features are determined from the historical analyte data.

At 1080, the selected model is trained based on the selected combination of features determined from the historical analyte data, and the clinical disease diagnoses associated with the historical analyte data.

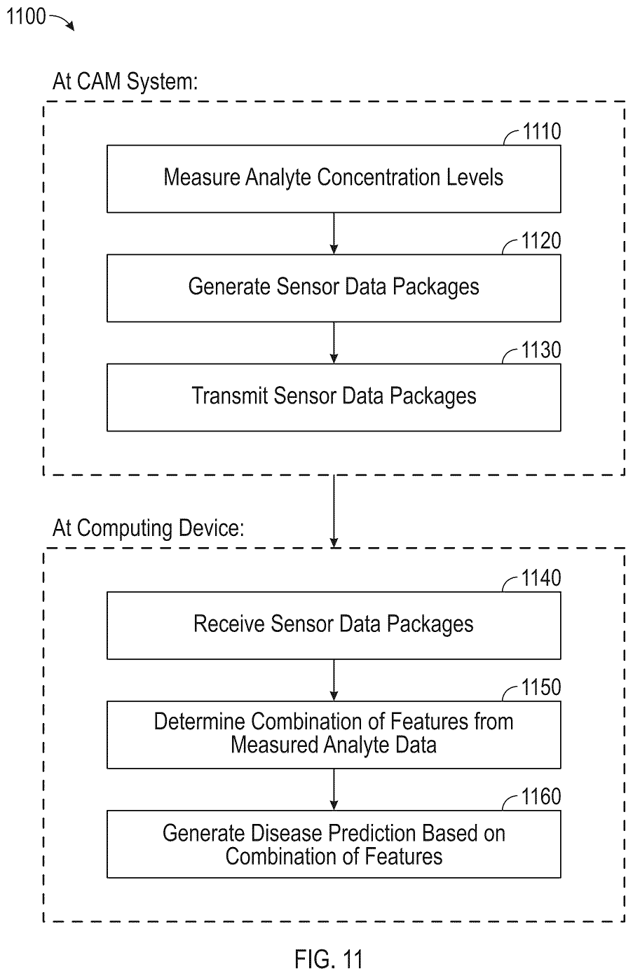

FIG. 11 depicts process flow diagram 1100 representing operations for predicting disease, in accordance with embodiments of the present disclosure.

In many embodiments, blocks 1110, 1120 and 1130 may be performed at CAM system 200, and blocks 1140, 1150 and 1160 may be performed at a computing device, such as network computing device 142 or one of display devices 150. In certain embodiments, blocks 1110, 1120, 1130, 1150 and 1160 may be performed at CAM system 200, and block 1140 may be performed at one of display devices 150, in accordance with the processes described above.

At block 1110, one or more analyte concentration levels are measured by an analyte sensor.

At block 1120, sensor data packages are generated based on the measured analyte concentration levels. The sensor data packages include, inter alia, measured analyte data. In certain embodiments, the measure analyte data includes the measured analyte concentration levels with associated time stamps.

At block 1130, the sensor data packages are transmitted to a computing device, such as display device 150, network computing device 142, etc.

At block 1140, the sensor data packages are received.

At block 1150, an analyte feature combination is determined from the measured analyte data.

At block 1160, the disease prediction is generated based on the analyte feature combination. In certain embodiments, the analyte feature combination is provided to a trained diagnostic model, which generates the disease prediction based on the analyte feature combination. In other embodiments, the analyte feature combination is provided to a trained screening model, which generates a disease “predisposition” prediction based on the analyte feature combination.

### Example Clauses

Implementation examples are described in the following numbered clauses:

Clause 1: A method for predicting a disease, the method comprising generating biased analyte data by adding analyte sensor bias to historical analyte data; associating the biased analyte data with clinical disease diagnoses associated with the historical analyte data; extracting features from the biased analyte data; for each model of a number of models: generating disease predictions based on different combinations of the features extracted from the biased analyte data, and evaluating the disease predictions based on the clinical disease diagnoses associated with the biased analyte data; and selecting a model and a combination of features based on a performance metric and a robustness metric, wherein the selected combination of features includes at least one feature with a high performance metric, and at least one feature with a high robustness metric that is relatively insensitive to analyte sensor bias.

Clause 2: The method of Clause 1, wherein the features include trend-related features, time-related and day-related features, variability and stability features, frequency-related features, and value-based features.

Clause 3: The method of Clause 1 or 2, wherein the number of models include bivariate models and multivariate models.

Clause 4: The method of Clauses 1, 2, or 3, wherein the selected model is a machine learning model.

Clause 5: The method of Clauses 1, 2, 3, or 4, wherein evaluating the disease predictions is based on a sensitivity and a specificity.

Clause 6: The method of Clauses 1, 2, 3, 4, or 5, further comprising determining the selected combination of features from the historical analyte data; and training the selected model based on the selected combination of features determined from the historical analyte data, and the clinical disease diagnoses associated with the historical analyte data.

Clause 7: The method of Clauses 1, 2, 3, 4, 5, or 6, further comprising at a continuous analyte monitoring (CAM) device: measuring analyte concentration levels of a user, generating, based on the measured analyte concentration levels, sensor data packages including measured analyte data, and transmitting the sensor data packages; and, at a computing device: receiving the sensor data packages, determining the selected combination of features from the measured analyte data, and generating a disease prediction based on the selected combination of features from the measured analyte data.

Clause 8: The method of Clause 7, wherein the sensor data packages are received by the computing device over a wireless connection; and the computing device is further configured to display the disease prediction in a graphical user interface (GUI).

Clause 9: The method of Clauses 1, 2, 3, 4, 5, 6, 7, or 8, wherein the disease is diabetes; the historical analyte data are historical glucose time series data; the clinical disease diagnoses are clinical diabetes diagnoses; the selected combination of features includes an autocorrelation mean feature and a set point frequency feature; the model is a bivariate linear regression (LR) model; and the measured analyte data are temperature-corrected glucose time series data.

Clause 10: A system for predicting a disease, the system comprising a continuous analyte monitoring (CAM) system, including: an analyte sensor configured to measure one or more analyte concentration levels, and a sensor electronics module (SEM) configured to: generate, based on the measured analyte concentration levels, sensor data packages including measured analyte data, and transmit the sensor data packages; and a computing device configured to: receive the sensor data packages, determine a combination of features from the measured analyte data, and generate a disease prediction based on the combination of features, wherein the selected combination of features includes at least one feature with a high performance metric, and at least one feature with a high robustness metric that is relatively insensitive to analyte sensor bias.

Clause 11: The system of Clause 10, wherein the sensor data packages are received by the computing device over a wireless connection; and the computing device is further configured to display the disease prediction in a graphical user interface (GUI).

Clause 12: The system of Clauses 10 or 11, further comprising a mobile computing device configured to: receive, from the CAM system over a wireless connection, the sensor data packages, transmit, to the computing device over a network, the sensor data packages, receive, from the computing device over the network, the disease prediction, and display, in a GUI, the disease prediction.

Clause 13: The system of Clauses 10, 11, or 12, wherein the computing device is configured to execute a model to generate the disease prediction; and the model is trained to predict the disease based on a combination of features determined from historical analyte data, and clinical disease diagnoses associated with the historical analyte data.

Clause 14: The system of Clause 13, wherein the model is a machine learning model.

Clause 15: The system of Clauses 10, 11, 12, 13, or 14, wherein the disease is diabetes; the analyte sensor is a glucose sensor; the measured analyte data are temperature-corrected glucose time series data; the trend-related feature is an autocorrelation mean feature; and the frequency-related feature is a set point frequency feature.

The many features and advantages of disclosure are apparent from detailed specification, and, thus, it is intended by appended claims to cover all such features and advantages of disclosure which fall within scope of disclosure. Further, since numerous modifications and variations will readily occur to those skilled in art, it is not desired to limit disclosure to exact construction and operation illustrated and described, and, accordingly, all suitable modifications and equivalents may be resorted to that fall within scope of disclosure.

What is claimed is:

1. A system for predicting a disease, the system comprising:
a continuous analyte monitoring (CAM) system, including:
an analyte sensor configured to measure one or more analyte concentration levels, and
a sensor electronics module (SEM) configured to:
generate, based on the measured analyte concentration levels, sensor data packages including measured analyte data, and
transmit the sensor data packages; and
a computing device configured to:
receive the sensor data packages,
determine a combination of features from the measured analyte data, and
generate a disease prediction based on the combination of features.

2. The system of claim 1, wherein:
the sensor data packages are received by the computing device over a wireless connection; and
the computing device is further configured to display the disease prediction in a graphical user interface (GUI).

3. The system of claim 1, further comprising:
a mobile computing device configured to:
receive, from the CAM system over a wireless connection, the sensor data packages,
transmit, to the computing device over a network, the sensor data packages,
receive, from the computing device over the network, the disease prediction, and display, in a GUI, the disease prediction.

4. The system of claim 1, wherein:
the computing device is configured to execute a model to generate the disease prediction; and
the model is trained to predict the disease based on a combination of features determined from historical analyte data, and clinical disease diagnoses associated with the historical analyte data.

5. The system of claim 4, wherein the combination of the features determined from the measured analyte data and the combination of the features determined from the historical analyte data include the same type of features.

6. The system of claim 5, wherein the model is a machine learning model.

7. The system of claim 1, wherein the features include trend-related features, time-related and day-related features, variability and stability features, frequency-related features, and value-based features.

8. The system of claim 7, wherein:
at least one feature has a high performance metric; and
at least one feature has a high robustness metric that is relatively insensitive to analyte sensor bias.

9. The system of claim 8, wherein the combination of features includes at least one trend-related feature and at least one frequency-related feature.

10. The system of claim 9, wherein:
the disease is diabetes;
the analyte sensor is a glucose sensor;
the measured analyte data are temperature-corrected glucose time series data;
the trend-related feature is an autocorrelation mean feature; and
the frequency-related feature is a set point frequency feature.

11. A method for predicting a disease, the method comprising:
at a network computing device:
generating biased analyte data by adding analyte sensor bias to historical analyte data;
associating the biased analyte data with clinical disease diagnoses associated with the historical analyte data;
extracting features from the biased analyte data;
for each model of a number of models:
generating disease predictions based on different combinations of the features extracted from the biased analyte data, and
evaluating the disease predictions based on the clinical disease diagnoses associated with the biased analyte data; and
selecting a model and a combination of features based on a performance metric and a robustness metric.

12. The method of claim 11, wherein the features include trend-related features, time-related and day-related features, variability and stability features, frequency-related features, and value-based features.

13. The method of claim 12, wherein the selected combination of features includes:
at least one feature with a high performance metric; and
at least one feature with a high robustness metric that is relatively insensitive to analyte sensor bias.

14. The method of claim 13, wherein the number of models include bivariate models and multivariate models.

15. The method of claim 14, wherein the selected model is a machine learning model.

16. The method of claim 15, wherein evaluating the disease predictions is based on a sensitivity and a specificity.

17. The method of claim 11, further comprising:
at the network computing device:
determining the selected combination of features from the historical analyte data; and
training the selected model based on the selected combination of features determined from the historical analyte data, and the clinical disease diagnoses associated with the historical analyte data.

18. The method of claim 17, further comprising:
at a continuous analyte monitoring (CAM) device:
measuring analyte concentration levels of a user,
generating, based on the measured analyte concentration levels, sensor data
packages including measured analyte data, and
transmitting the sensor data packages; and
at a computing device:
receiving the sensor data packages,
determining the selected combination of features from the measured analyte data, and
generating a disease prediction based on the selected combination of features from the measured analyte data.

19. The method of claim 18, wherein:
the sensor data packages are received by the computing device over a wireless connection; and
the computing device is further configured to display the disease prediction in a graphical user interface (GUI).

20. The method of claim 19, wherein:
the disease is diabetes;
the historical analyte data are historical glucose time series data;
the clinical disease diagnoses are clinical diabetes diagnoses;
the selected combination of features includes an autocorrelation mean feature and a set point frequency feature;
the model is a bivariate linear regression (LR) model; and
the measured analyte data are temperature-corrected glucose time series data.

21. A method for predicting a disease, the method comprising:
at a network computing device:
extracting features from historical analyte data having inherent analyte sensor bias;
for each model of a number of models:
generating disease predictions based on different combinations of the features extracted from the historical analyte data, and
evaluating the disease predictions based on clinical disease diagnoses associated with the historical analyte data; and
selecting a model and a combination of features based on a performance metric and a robustness metric.

22. The method of claim 21, wherein the features include trend-related features, time-related and day-related features, variability and stability features, frequency-related features, and value-based features.

23. The method of claim 22, wherein the selected combination of features includes:
at least one feature with a high performance metric; and
at least one feature with a high robustness metric that is relatively insensitive to analyte sensor bias.

24. The method of claim 23, wherein the number of models include bivariate models and multivariate models.

25. The method of claim 24, wherein the selected model is a machine learning model.

26. The method of claim 25, wherein evaluating the disease predictions is based on a sensitivity and a specificity.
